(19) United States
(12) Patent Application Publication
(10) Pub. No.: US 2015/0010992 A1
(43) Pub. Date: Jan. 8, 2015

(54) OPTICAL READER SYSTEMS AND LATERAL FLOW ASSAYS

(71) Applicant: QuantRx Biomedical Corporation, Tualatin, OR (US)

(72) Inventors: William Fleming, Tualatin, OR (US); Robert Buck, Fairview, OR (US); Ray Miracle, Gresham, OR (US); Dan Morrow, Redwood City, CA (US); Louis Dietz, Mountain View, CA (US); Scott Myrick, Portland, OR (US)

(73) Assignee: QuantRx Biomedical Corporation

(21) Appl. No.: 14/336,731

(22) Filed: Jul. 21, 2014

Related U.S. Application Data
(63) Continuation of application No. 13/978,116.
(60) Provisional application No. 61/366,130, filed on Jul. 20, 2010.

(51) Int. Cl. B01L 9/00; G01N 33/78; G01N 33/558; B01L 3/00

(52) U.S. Cl. CPC B01L 9/52; B01L 3/5023; G01N 33/78; G01N 33/558; B01L 2300/0825
USPC 435/287.2; 422/554; 422/69

(57) ABSTRACT

Systems and methods for determining the presence and/or amount of analytes in a fluid sample are described.

### CROSS-REFERENCE TO RELATED APPLICATIONS

This application is a continuation of U.S. application Ser. No. 13/978,116, filed on Jul. 2, 2013, which is the U.S. National Stage of International Application No. PCT/US2011/044631, filed Jul. 20, 2011, which was published in English under PCT Article 21(2), which in turn claims the benefit of U.S. Provisional Application No. 61/366,130, filed Jul. 20, 2010. The priority applications are incorporated by reference herein in their entirety.

### FIELD

This disclosure relates generally to the detection of analytes in various diagnostic test devices.

### BACKGROUND

Analytical tests have been developed for the routine identification or monitoring of physiological and pathological conditions (e.g., pregnancy, cancer, endocrine disorders, infectious diseases) using different biological samples (e.g., urine, serum, plasma, blood, saliva), and for analysis of environmental samples (e.g., natural fluids and industrial plant effluents) for instance for contamination. Many of these tests are based on the highly specific interactions between specific binding pairs. Furthermore, many of these tests involve devices (e.g., solid phase, lateral-flow test strips, flow-through tests) with one or more of the members of a binding pair attached to a mobile or immobile solid phase material such as latex beads, glass fibers, glass beads, cellulose strips or nitrocellulose membranes. However, currently available analytical tests suffer from various deficiencies including, for example, test sensitivity, test variability (even among analytical tests of a common lot), cost, and ease of use.

### SUMMARY

The following embodiments relate to systems and methods for determining the presence and/or amount of analytes in a fluid sample. The methods and devices disclosed herein can be used to detect analytes in various types of fluid, including biological specimens (such as blood, serum, plasma, urine, saliva, milk) and environmental samples (such as industrial plant effluent or natural fluids). Results from the methods and devices disclosed herein can be positively read directly from the assay device by visual inspection or using an electronic reader, such as those disclosed herein.

In a first embodiment, an optical reader for performing a diagnostic test on a test sample is provided. The reader comprises a cassette receiving member, an excitation member, and an imaging system. The cassette receiving member is configured to receive at least one cassette comprising a lateral flow strip with a test sample received thereon. The excitation member is positioned to direct excitation energy towards the at least one cassette when the at least one cassette is received by the cassette receiving member. The excitation member comprises a flashlamp that is configured to emit a single flash for each diagnostic test. The imaging system is configured to capture an image of a viewing area. The viewing area comprises at least a portion of the at least one cassette.

In another embodiment, an optical reader for performing a diagnostic test on a test sample is provided that comprises a cassette receiving member, an excitation member, and a CMOS sensor. The cassette receiving member is configured to receive at least one cassette comprising a lateral flow strip with a test sample received thereon. The excitation member is positioned to direct excitation energy towards the at least one cassette when the at least one cassette is received by the cassette receiving member. The CMOS sensor is configured to capture an image of a viewing area, with the viewing area comprising at least a portion of the at least one cassette.

In another embodiment, a method of performing a diagnostic test is provided. The method comprises positioning a cassette in an optical reader, the cassette comprising at least one lateral flow strip; directing a single flash of excitation energy toward an exposed portion of the at least one lateral flow strip in the cassette; and capturing an image from a viewing area using an imaging system, with the viewing area comprising the exposed portion.

In another embodiment, a cassette is provided. The cassette includes a housing comprising a top member and a bottom member, and a lateral flow strip receiving area located between the top and bottom members. The housing comprises one or more biased members that have a fixed end and a free end, with the free end being configured to contact at least a portion of a lateral flow strip when the lateral flow strip is positioned in the lateral flow strip receiving area.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates an exemplary optical reader system, with portions of the optical reader system shown as transparent.

FIG. 2 illustrates a side view of an exemplary optical reader system, with portions of the optical reader system removed for clarity.

FIG. 3 is a top perspective view of the optical reader system of FIG. 2.

FIG. 4 is a side perspective view of the optical reader system of FIG. 2.

FIG. 5 is an exemplary system block diagram for an optical reader system.

FIG. 6 is a schematic of a system for exciting a detection zone and directing emitted fluorescent light at an imaging system.

FIG. 7 is a schematic of another system for exciting a detection zone and directing emitted fluorescent light at an imaging system.

FIG. 8 illustrates a lateral flow immunoassay test strip for use with an optical reader system.

FIG. 9 illustrates a cassette member for housing a lateral flow immunoassay test strip such as that shown in FIG. 8.

FIG. 10 illustrates a cassette member for housing a lateral flow immunoassay test strip such as that shown in FIG. 8.

FIG. 11A illustrates a graph of a TRF signal and a background signal over time.

FIG. 11B illustrates a graph of a reader signal and flash energy over flash duration.

FIG. 12 illustrates a photograph capture of the entire current (Trace A) and light events (Trace B).

FIG. 13 illustrates a plurality of lateral flow strips and a detection zone that comprises portions of the different lateral flow strips.

FIG. 14 illustrates a cassette configured to hold at least two different lateral flow strips for insertion into a reader system.

FIG. 15 illustrates a plurality of lateral flow strips and detection zone that comprises portions of the different lateral flow strips.

FIG. 16 illustrates a cassette that comprises a detection zone that includes a portion of a lateral flow strip and a bar code.

FIG. 17 illustrates a series of views of a graphical user interface of LCD touchscreen 18 during operation of various testing procedures.

FIG. 18 is a flow chart illustrating various steps that can be performed by a reader system.

FIGS. 19 and 20 are tables of the results of test and reference line quantization of the TSH test of Example 1.

FIG. 21 is a graph of the results of the TSH test of Example 1.

FIG. 22 is a schematic view of a cassette that has one or more biased members for securing a lateral flow strip in the cassette.

FIG. 23 is a schematic view of the cassette of FIG. 22.

FIG. 24 is a view of a bottom part of a cassette, which contains a pair of biased members.

FIG. 25 is a bottom view of a cassette that comprises a pair of biased members.

FIG. 26 is a top view of a top part of a cassette that houses a lateral flow strip.

FIG. 27 is a side view of the top part of FIG. 26.

FIG. 28 is a cross-sectional view of the top part of FIG. 26, taken about line A-A.

FIG. 29 is a bottom view of the top part of FIG. 26.

FIG. 30 is a bottom view of a cassette that comprises a plurality of biased members and a plurality of later flow strips.

FIG. 31 is a graph of the results of the FT4 test of Example 2.

### DETAILED DESCRIPTION

Various embodiments of support members and methods of their use are disclosed herein. The following description is exemplary in nature and is not intended to limit the scope, applicability, or configuration of the invention in any way. Various changes to the described embodiment may be made in the function and arrangement of the elements described herein without departing from the scope of the invention.

As used in this application and in the claims, the singular forms “a,” “an,” and “the” include the plural forms unless the context clearly dictates otherwise. Additionally, the term “includes” means “comprises.” Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

### Definitions

Analyte: An atom, molecule, group of molecules or compound of natural or synthetic origin (e.g. drug, hormone, enzyme, growth factor antigen, antibody, hapten, lectin, apoprotein, cofactor) sought to be detected or measured that is capable of binding specifically to at least one binding partner (e.g. drug, hormone, antigen, antibody, hapten, lectin, apoprotein, cofactor).

The various embodiments disclosed herein can be practiced with assays for virtually any analyte. The analytes may include, but are not limited to antibodies to infectious agents (such as HIV, HTLV, Helicobacter pylori, hepatitis, measles, mumps, or rubella), cocaine, benzoylecgonine, benzodizazpine, tetrahydrocannabinol, nicotine, ethanol theophylline, phenyloin, acetaminophen, lithium, diazepam, nonryptyline, secobarbital, phenobarbitol, methamphetamine, theophylline, testosterone, estradiol, estriol, 17-hydroxyprogesterone, progesterone, thyroxine, thyroid stimulating hormone, follicle stimulating hormone, luteinizing hormone, transforming growth factor alpha, epidermal growth factor, insulin-like growth factor I and II, growth hormone release inhibiting factor, IGA and sex hormone binding globulin; and other analytes including antibiotics (e.g., penicillin), glucose, cholesterol, caffeine, cotinine, corticosteroid binding globulin, PSA, or DHEA binding glycoprotein.

Analytes vary in size. Merely by way of example, small molecule analytes may be, for instance, <1.0 nm (such as cotinine or penicillin, each with a molecular weight of less than about 1,000 Daltons). However, analytes may be larger than this, including for instance immunoglobulin analytes (such as IgG, which is about 8 nm in length and about 160,000 Daltons).

Analyte analog: A modified analyte that has structural similarity to the unmodified analyte and can bind to at least one analyte binding partner. In certain embodiments of the invention, the analyte analog is an analyte-tracer conjugate, for instance a detectable analyte-tracer conjugate.

Label: Any molecule or composition bound to an analyte, analyte, analog or binding partner that is detectable by spectroscopic, photochemical, biochemical, immunochemical, electrical, optical or chemical means. Examples of labels, including enzymes, colloidal gold particles, colored latex particles, have been disclosed (U.S. Pat. Nos. 4,275,149; 4,313,734; 4,373,932; and 4,954,452, each incorporated by reference herein).

The attachment of a compound (e.g., an analyte) to a label can be through covalent bonds, adsorption processes, hydrophobic and/or electrostatic bonds, as in chelates and the like, or combinations of these bonds and interactions and/or may involve a linking group.

Lateral flow device: Devices that include bibulous or non-bibulous matrices capable of transporting analytes and reagents to a pre-selected site. Many such devices are known, in which the strips are made of nitrocellulose, paper, cellulose, and other bibulous materials. Non-bibulous materials can be used, and rendered bibulous by applying a surfactant to the material.

Lateral flow strip: A test strip used in lateral flow chromatography, in which a test sample fluid, suspected of containing an analyte, flows (for example by capillary action) through the strip (which is frequently made of materials such as paper or nitrocellulose). The test fluid and any suspended analyte can flow along the strip to a detection zone in which the analyte (if present) interacts with a detection agent to indicate a presence, absence and/or quantity of the analyte.

### Optical Reader System

The optical reader system described herein generally comprises an opto-fluorescent instrument with an integrated software analysis capability. The instrument can be a stand-alone instrument capable of providing a diagnostic result to a user. In one embodiment disclosed herein, by utilizing the principles of time resolved fluorescence (TRF), the reader system can detect a fluorescent signal emitted by a reporter (e.g., a tracer molecule such as an enzyme, fluorophore, or other molecule known to produce a detectable and/or measurable product or signal) to determine the presence and/or amount of analyte in a sample. In some embodiments, as described in detail below, a lateral flow chromatography strip can be used in combination with the optical reader system to detect the presence and/or amount of various analytes.

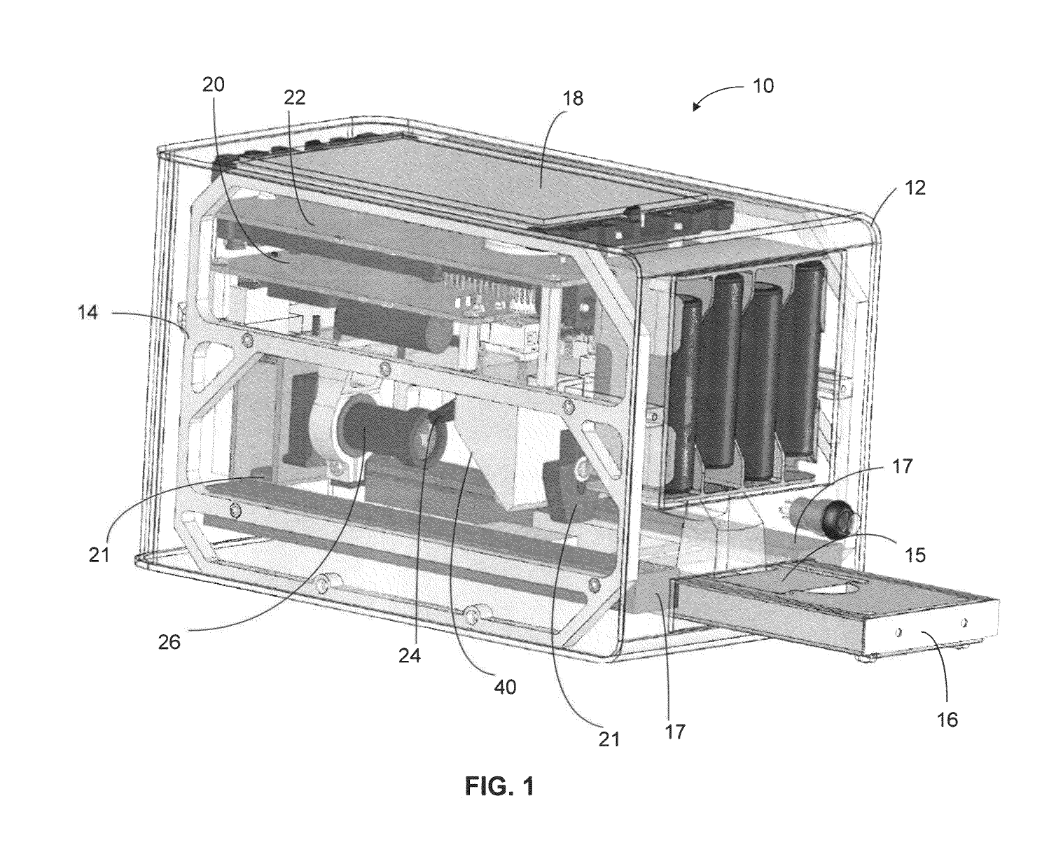

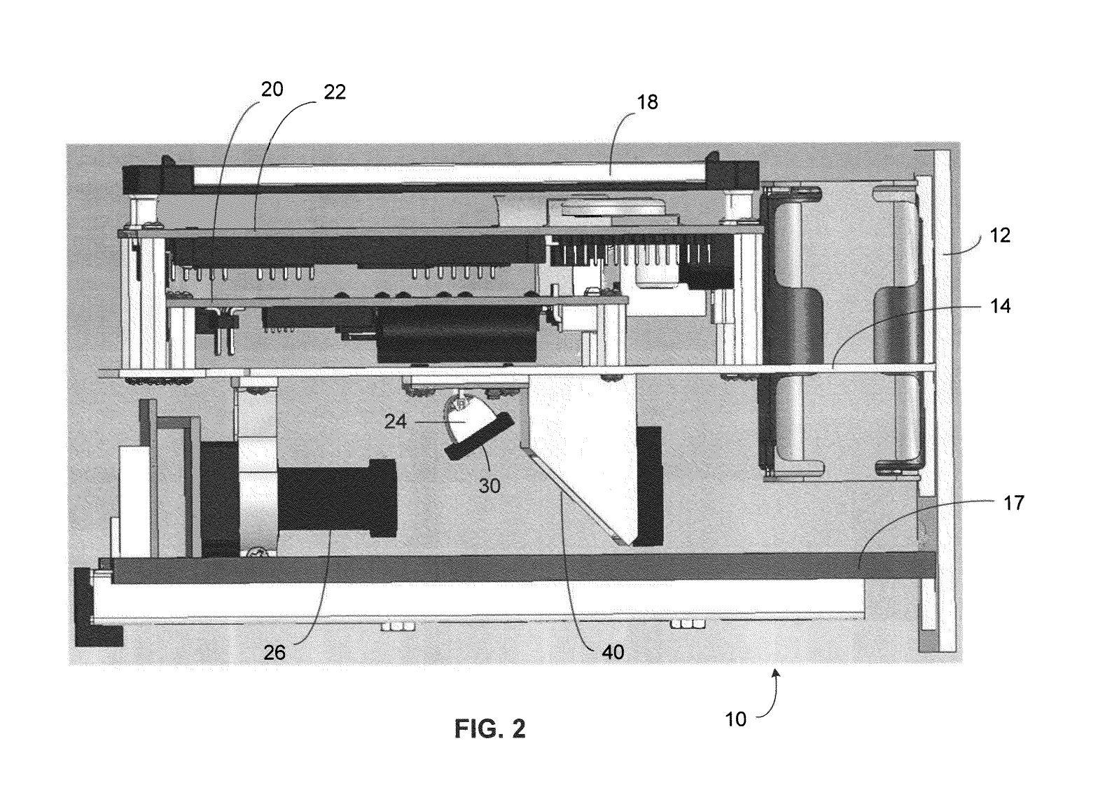

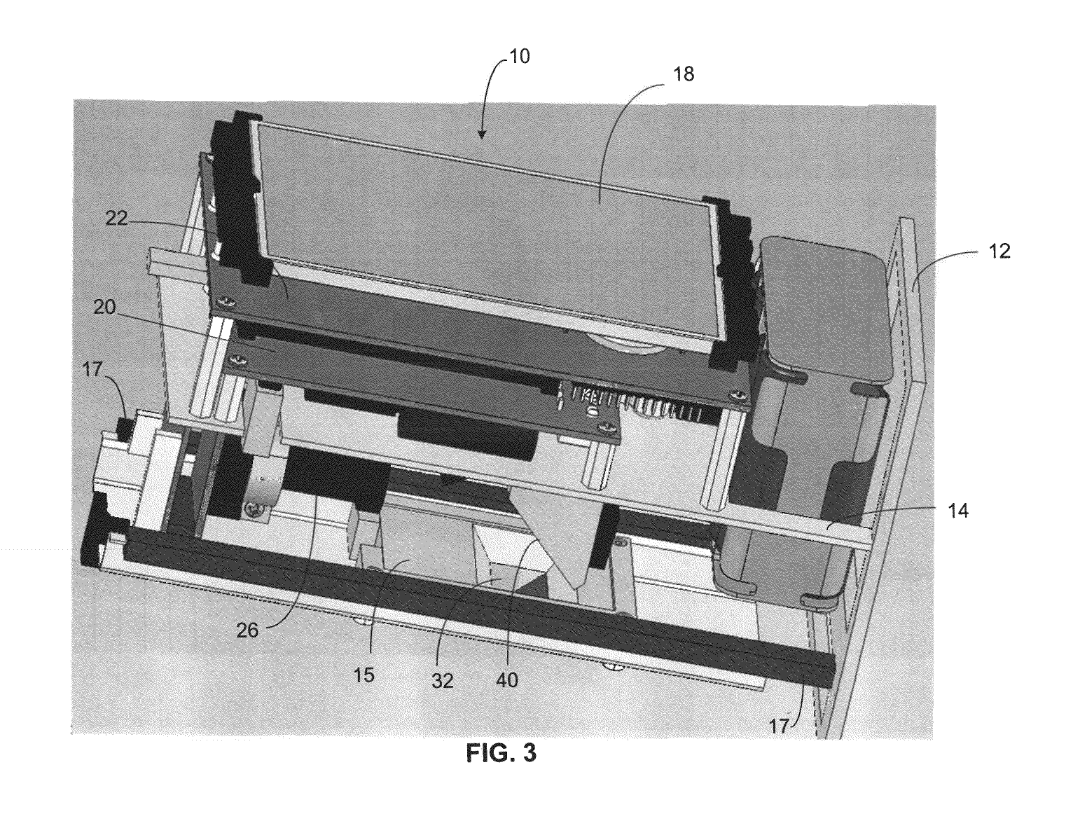

FIGS. 1-4 illustrate an optical reader system 10 comprising an imaging system for determining the presence and/or amount of analytes in a fluid sample. Optical reader system 10 comprises a housing 12 that contains an imaging system 26. To illustrate the internal structure and components of reader system 10, housing 12 is shown as transparent in FIG. 1 and portions of housing 12 have been removed from FIGS. 2-4.

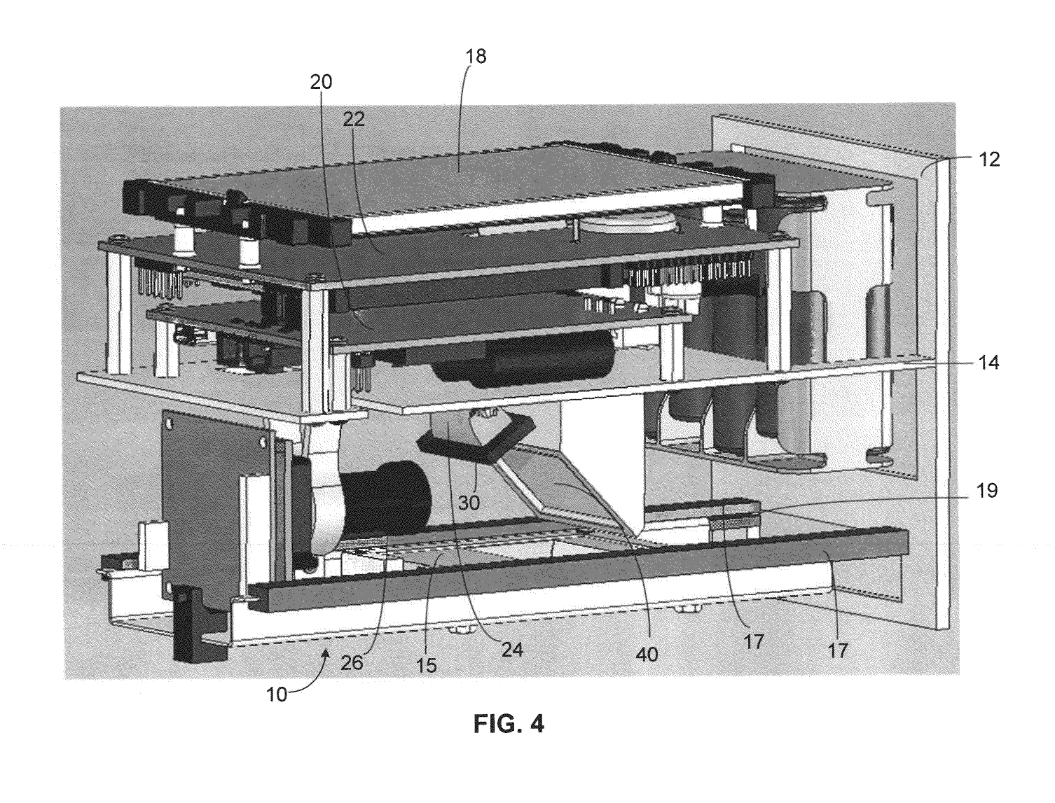

Housing 12 comprises a supporting structure 14 (e.g., a chassis or skeleton) for supporting imaging system 26 and other components (e.g., various optical and electrical components) within housing 12. Housing 12 can also include a receiving member 16 for receiving a sample on a substrate or other sample-carrying structure. Receiving member 16 can comprise, for example, a drawer that is moveable between a first open position for receiving a cassette 15 (FIG. 1), and a second closed position whereby cassette 15 is moved into housing 12 and positioned for analysis by reader system 10. First and second guide members 17 (e.g., runners) can be provided on opposing sides of the drawer to guide the drawer between the open and closed positions. Guide members 17 can have a slot 19 (FIG. 4) or other receiving portions for guiding and receiving the drawer as it moves from the open to the closed position. One or more optical position sensors 21 can be provided to determine whether the drawer and/or cassette are in the proper position for running a test using reader system 10.

In an alternate embodiment, instead of a drawer configured to receive a sample-carrying structure, receiving member 16 can comprise an opening into which a cassette or other sample-carrying structure can be directly received.

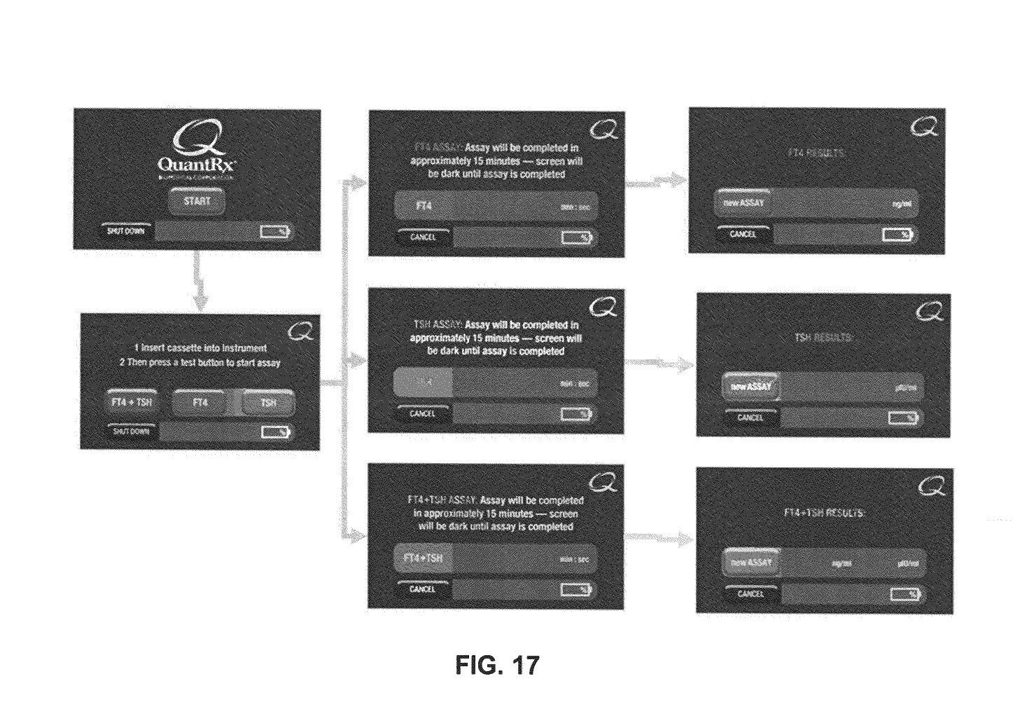

A display and input screen (e.g., an LCD touchscreen) 18 can be provided on a surface of housing 12. LCD touchscreen 18 can be configured to receive information from a user and to display information to the user about the status of reader system 10 and/or about an analytical test that can be or has been performed by reader system 10. Reader system 10 can be powered by batteries (e.g., batteries 19) and/or it can include a power plug for operating the device on power supplied from an external source, including, for example, AC power. FIG. 17 illustrates a series of views of a graphical user interface of LCD touchscreen 18 during operation of various testing procedures.

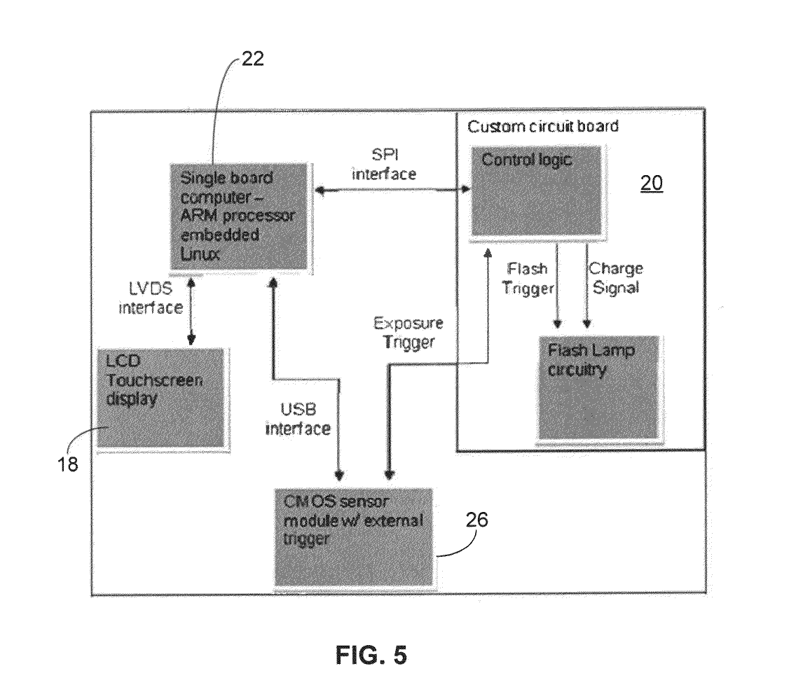

One or more circuit boards 20, 22 can be provided to control the operation of reader system 10 and display and receive information on LCD touchscreen 18. FIG. 5 illustrates an exemplary system block diagram of reader system 10. For example, as shown in FIG. 5, a first circuit board 20 can be configured to control the operation of an excitation member 24 (discussed in more detail below) and a second circuit board 22 can be configured to process information or data received from imaging system 26.

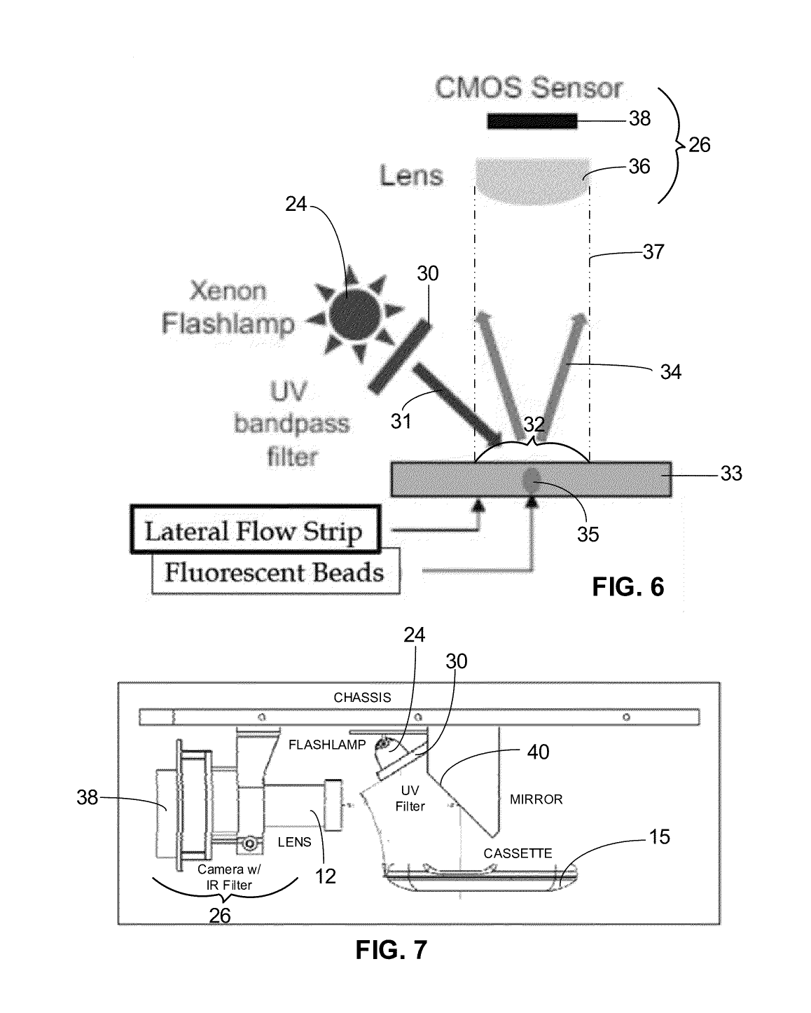

FIGS. 6 and 7 illustrate schematic representations of the operation of reader system 10. As shown in FIG. 6, excitation member 24 (e.g., a Xenon flashlamp) emits light 31 at a detection zone 32. In the illustrated embodiment, detection zone 32 comprises a portion of a lateral flow strip 33 that comprises one or more reporters (e.g., fluorescent beads 35) that emit fluorescent light 34 when illuminated by excitation member 24. Emitted fluorescent light 34 is directed towards imaging system 26, which can comprise at least one lens 36 and a CMOS sensor 38. Lens 36 can comprise multiple lenses configured to direct and focus light on the sensor 38. In one embodiment, lens 36 can comprise a TECHSPEC® Megapixel Finite Conjugate μ-Video™ Imaging Lens that includes several precision glass elements mounted in a compact aluminum housing. As discussed in more detail below, light from excitation member 24 can be directed through a filter 30 before impacting detection zone 32.

As shown in FIG. 6, imaging system 26 can have a field of view 37 that is capable of detecting the entire detection zone 32. As discussed in more detail below in other embodiments, field of view 37 can be sufficiently large to detect multiple detection zones. These multiple detection zones can include portions of multiple lateral flow strips 33 (or other analytical test members) and/or other viewable elements that contain information about the analytical tests being performed by the reader system.

It should be understood that various arrangements of the excitation member, detection zone, and imaging system are possible. For example, the imaging system and related optical elements can comprise multiple filters, lenses, and minors in one or more assemblies to focus an image of the detection zone on a sensor of the imaging system. Similarly, multiple optical components can be used to focus excitation light necessary for fluorescent assay analysis. For example, unlike the schematic view shown in FIG. 6, FIGS. 1-4 and 7 include an optical member 40 (e.g., a mirror) that redirects emitted fluorescent light 34 from the detection zone 32 towards imaging system 26.

### Imaging System

Conventional assay detection systems use non-imaging detectors such as photodiodes or photomultiplier tubes to measure the light emitted from the fluorescent or time resolved fluorescent tags. In contrast, the systems and methods disclosed herein generally include imaging systems. The imaging systems disclosed herein can greatly improve the amount of information that can be received, which increases the flexibility in the design of such assay systems. For example, in lateral flow immunoassays, there are typically multiple zones in which the optical signal needs to be measured. While multiple non-imaging detectors can be used to measure the signal from those zones or a single non-imaging detector can be scanned over the device to measure multiple zones, the resulting device is both complex and inflexible in that it can function only with the assay format for which it was designed. The imaging systems described herein, however, can be adapted for use with multiple assay formats by updating the image analysis software which is used to compute the results of the assay. In this manner, the imaging systems of the present disclosure are highly flexible and easily modifiable to perform multiple types of measurements simultaneously and/or in sequence.

Additionally, the imaging systems disclosed herein can have spatial resolution, which can be useful in performing quality checks on the assay device. For example, by using spatial resolution, the imaging systems disclosed herein can detect or adapt to imperfections in the operation of the assay device, making the system more robust and reliable.

Imaging system 26 can comprise, for example, a CMOS or CCD image sensor, or a 2-dimensional array of photosensitive detectors such as photodiodes, avalanche photodiodes, photomultipliers tubes, or other similar elements. By acquiring an image of the detection zone of the assay device, and using software to analyze the image, many advantages are realized. For example, many lateral-flow assay devices incorporate multiple fluorescing regions, such as test zones and calibrations zones, both of which can be imaged simultaneously by a single imaging sensor. Also, since the system microcontroller or microprocessor can analyze the image to automatically locate and measure the assay detection zones, the mechanical tolerances of the system and the assay substrate may be increased, allowing for a lower-cost device. In addition, the imaging sensor can be used to detect variation in the assay devices, (such as flow abnormalities in lateral flow assay devices) allowing the microprocessor to detect and/or account for error conditions. Moreover, as discussed in more detail herein, the field of view of the imaging sensor can be sufficiently large to image and measure several different areas in a detection zone at once, including, for example, bar code information and/or multiple assays arranged in different locations in the sample cassette (as in FIG. 5 below). The imaging of the detection zone can be done by a single image exposure, or using multiple exposures to optimize the imaging and TRF parameters (such as exposure time, flash duration, delay between flash and image exposure, etc.).

To improve the accuracy of measurement results, it can be useful to measure and compare the signal from multiple locations in the image field of view (FOV), resulting in a quantitative or semi-quantitative measurement result. For example, FIG. 13 illustrates an image of two side-by-side lateral flow immunoassay test strips. The signal (TRF or fluorescent light intensity) can be measured in two zones or “bands” on each test strip to obtain, for example, a ratio of the Test band to the Reference band (T/R ratio). This ratio can be used to normalize the response of the assay to several sources of error or uncertainty that would otherwise make the measurement much less accurate. Typically, this T/R ratio could be used together with a lot-specific calibration curve to predict the analyte concentration. For this ratio to be measured accurately, it is useful for the imaging system to have uniform response to fluorescent signals everywhere in the FOV view of the system.

However, most imaging systems do not have perfectly uniform illumination and detector sensitivity across the entire FOV. Since the measured fluorescent signals vary with both illumination and imaging system and detector sensitivity, it can be useful to measure and correct for such typical variations. In digital imaging, this process is typically called “flat-fielding,” and involves compensating for different gains and dark-current offsets for each pixel in the image FOV. This compensation can be relatively straightforward to perform using linear algebra, and a typical approach is to subtract a “dark reference” image from the measured image, and then divide the result by a “white reference” image, which is an image taken of a bright (but non-saturating) uniform object filling the FOV.

In imaging TRF, the dark reference image can be measured by disabling the illumination source while acquiring the dark reference image. Acquiring the white reference image, however, can be more complex, since it can be difficult to provide a calibration target that will emit uniform TRF signal over a large area, filing the FOV. In one embodiment of the present disclosure, this problem is resolved by replacing the sample with a calibration target that is a clean, uniform piece of material positioned at or adjacent the location of the sample. The material can scatter illumination energy (e.g. from the flashlamp) toward the imaging optics that form an image on the image sensor. In this manner, the white reference process can correct for both illumination variation and imaging system sensitivity versus position in the FOV.

Because the scattered light that forms the white reference image is at a wavelength that is different from the TRF emission, this technique works best if the imaging system sensitivity is the same or largely the same at the illumination wavelength and the TRF emission wavelength. Such a system can be accomplished by, for example, designing the optical system so that chromatic aberrations arc controlled and similar between these two different wavelengths. Scattered illumination light can also be many orders of magnitude brighter than the TRF emission; therefore it can be useful to provide a means to attenuate the white reference signal so that the detector is not saturated, without modifying the illumination pattern across the FOV. In one embodiment, this can be accomplished by reducing the intensity and duration of the flashlamp, and by increasing the delay between the flash and the image acquisition, and also by reducing the exposure time of the image sensor, or some combination of all three of these approaches.

### Outputs and Displays from the Optical Reader System

The results of a diagnostic test run by the optical reader systems described herein can be viewed in a number of ways. For example, the results can be displayed on the display screen of the device, printed, and/or delivered to another system for viewing or printing. The information relating to the results of a diagnostic test from the device to a printer or other system can be delivered via a wired or wireless system.

In a wired system, a universal serial bus (USB) port or other such output port can be provided to connect the system to an external device to output information from the optical reader system to the external device. Similarly, the establishment of communication between the optical reader system and an external device can allow for information to be exchanged from the external device to the optical reader system. Such information can include, for example, system upgrades and the delivery of additional or modified software for running various diagnostic tests on the optical reader system.

### Lateral Flow Device

As discussed above, in some embodiments, the detection zone imaged by the reader systems described herein can be a portion of one or more test strips in a lateral flow device. Using a lateral flow device, a test sample fluid that is suspected of containing an analyte can be allowed to flow (for example by capillary action) through the test strip (which is frequently made of materials such as paper or nitrocellulose). The test fluid and any suspended analyte can flow along or through the strip to a detection zone in which the analyte (if present) interacts with a detection agent to indicate a presence, absence and/or quantity of the analyte.

In one embodiment, the test strip can be configured to detect human thyroid stimulating hormone (hTSH) in human plasma or serum as an aid in the assessment, diagnosis, and treatment of a hypothyroid status and/or condition. Thyroid stimulating hormone (TSH) is released by the pituitary gland and exerts its action upon the thyroid gland, which in turn maintains normal blood levels of the iodothyronines T3 and T4 (thyroxine) in the body. TSH levels are controlled by thyrotropin releasing hormone and through negative feedback of iodothyronines. When thyroid hormone production is impaired, as in primary hypothryroidism, the levels of TSH in the blood rise. Conversely, TSH levels may be reduced when thyroid hormone production is low, as in secondary or tertiary hypothyroidism. In the case of hyperthyroidism, TSH levels are typically subnormal. Measuring blood levels of TSH provides a way to conveniently screen for thyroid disease and to monitor patients receiving TSH replacement therapy.

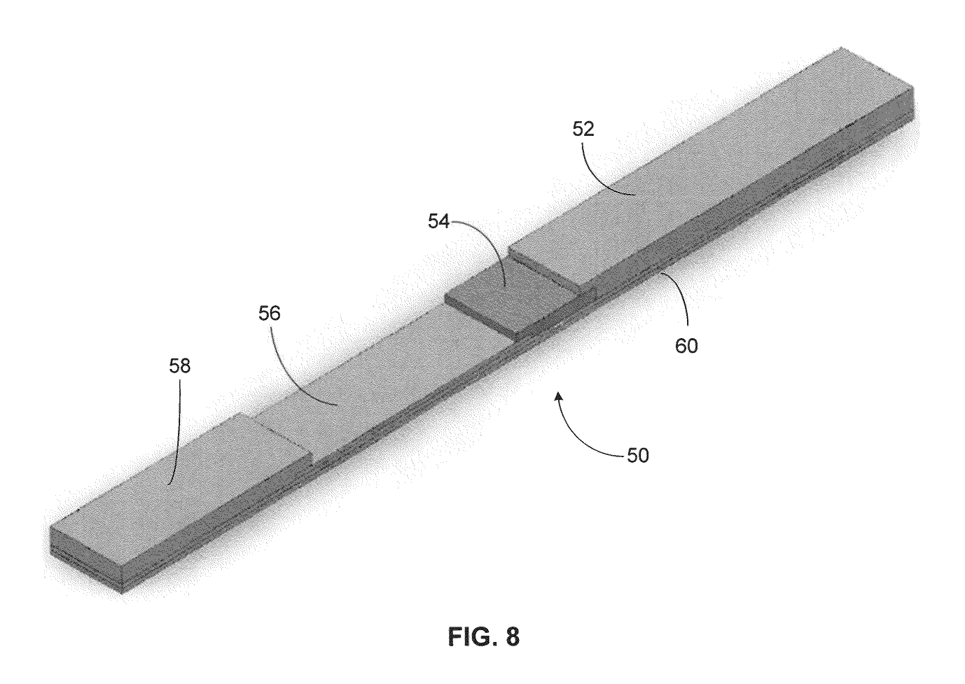

Referring to FIG. 8, a test strip 50 can comprise a sample pad 52, a conjugate pad 54, a nitrocellulose membrane 56, and an absorbent pad 58. The membrane-based immunoassay strip can have a test line and a reference/control line provided on the membrane (e.g., by spraying) at specific positions. The test line reagent can comprise an anti-beta TSH antibody and reference/control line can comprise immobilized streptavidin. After treating conjugate pad 54 with a buffer, a reference conjugate of Europium labeled BSA-Biotin can be provided on conjugate pad 54 (e.g., by spraying). A backing 60 can be applied to the membrane 56 and the entire test strip 50 can be inserted into a structural support, such as a cassette.

### Example 1

### TSH Test with CMEU-Latex-TSH Antibody Conjugate and Reader System Reagents

A CMEU Latex-TSH antibody conjugate was activated using as follows: To 0.20 ml of EDAC prepared CMEU Latex (Seradyne), is added 800 ul of 10 mM Borate buffer. Antibody (Medix Anti TSH 5405) is added. The mixture was agitated, for 3 hrs to covalently bind the antibody. Particles are washed three times in 10 mM borate and spun down by centrifugation, resuspended in conjugate dilution buffer. The CMEU conjugate is sprayed onto blocked glass at a rate of 4 ul/cm and dried to form the conjugate strip. A second conjugate for the reference line is prepared using CMEU Biotin and is manufactured in the same manner.

Nitrocellulose striping was accomplished with the TSH conjugate (Medix Anti TSH 5409) in the primary/test zone (ug/ml at 1.0 ul/cm) and streptavidin to the secondary/reference at (30 ug/ml at 1.0 ul/cm).

TSH (BiosPacific TSH-Ag J11030015) calibration test solutions were prepared in a human plasma or serum matrix by spiking with TSH dissolved in 10 mM Phosphate to levels of 1.0, 2.5, 4.0, 7.5, and 10.0 uIU/ml TSH. The calibration test solution's TSH levels were determined using an EIA Kit from Diagnostic Automation, Inc. Cat # 3122-18 and found to be acceptable.

### Construction of Test Strips

A 15 mm by 30 cm strip of nitrocellulose was affixed to a 50 mm×300 mm adhesive coated vinyl backing.

One line of a 1.0 ug/ml solution of the anti TSH was applied to (sprayed on) at a rate of 1 ul/cm to the nitrocellulose membrane (primary capture/test) to produce a 1 mm band. One line of a 30 ug/ml streptavidin solution was applied to (sprayed on) the nitrocellulose membrane (reference line). The nitrocellulose membrane was dried for 12 hours at 37.degrees C.

A sample pad sheet (Whatman Cytosep 1663) was wetted by immersion in a blocking solution then dried overnight. The dried sample pad was then laminated onto the lower portion of the strip. A cellulose pad was laminated to the upper portion of the strip to act as an absorbent reservoir.

The CMEU Latex TSH-antibody/CMEU biotin conjugate strip is then inserted between the membrane and the sample pad with a 2 mm overlap onto the membrane.

The completed card was cut into 4.3 mm strips. Strips were inserted into custom plastic cassettes with a hole/well located in the sample application area and a window in the test reading area.

### Test Procedure

A 60 ul aliquot sample dilution buffer and 60 ul aliquot of TSH test solution was added pre-mixed and then added to the sample well of each test strip device. The test strips were allowed to develop for 15 minutes.

### Results

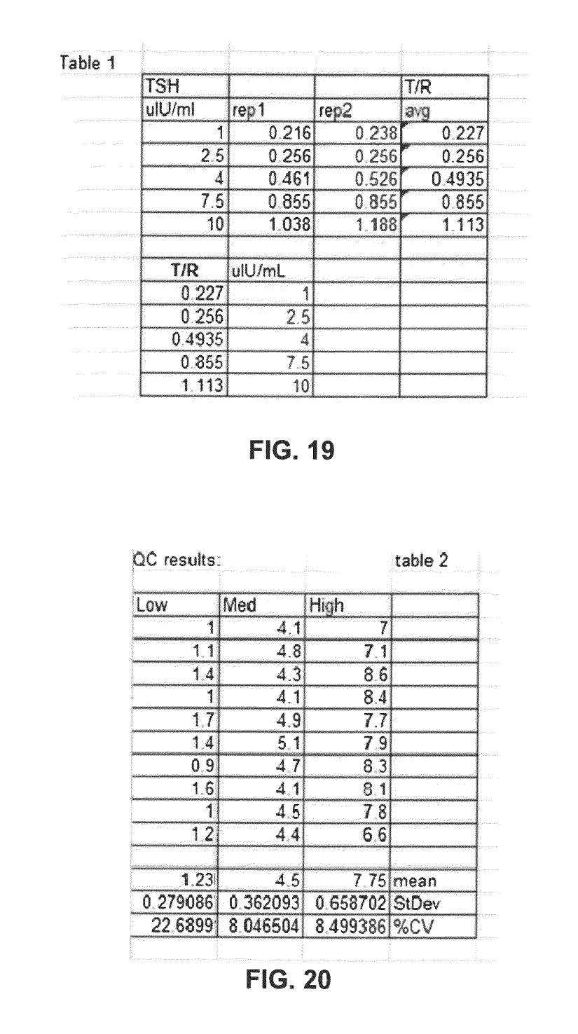

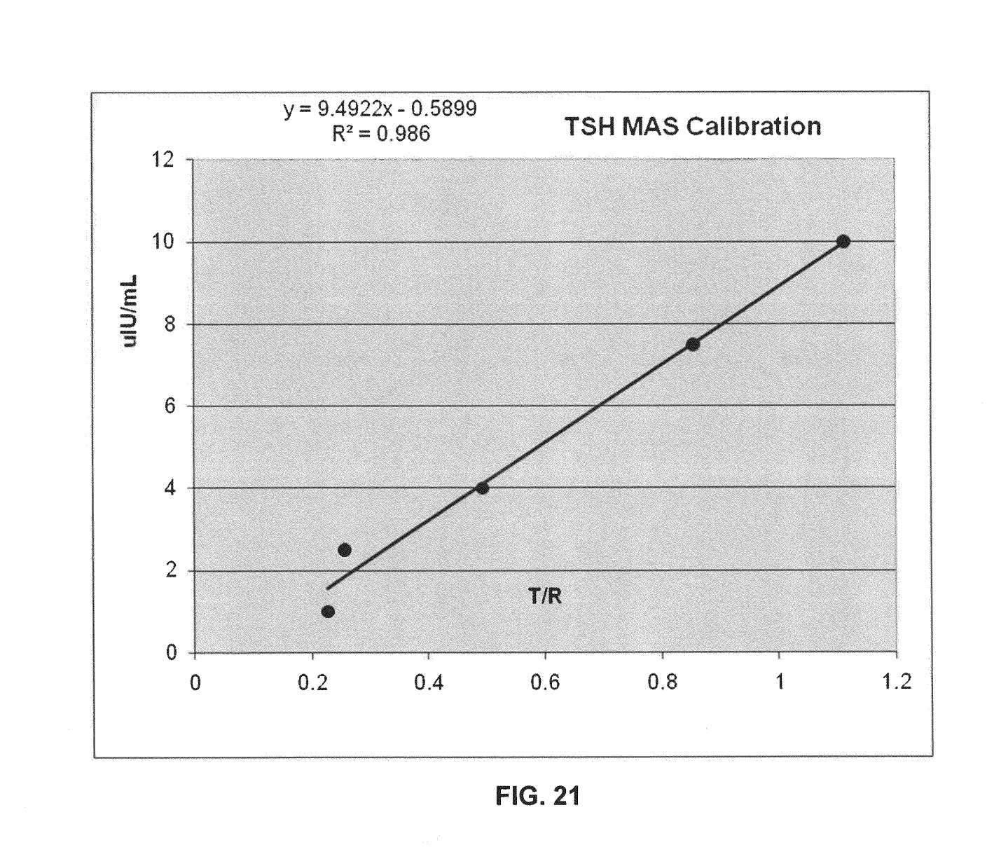

The test strips were then quantitated using the reader system to obtain values for the area of the reference and test lines. The T/R ratio was calculated from the areas, and the calibration curve was established. The results are depicted in Tables 1 and 2 shown in FIGS. 19 and 20, and the graph shown in FIG. 21.

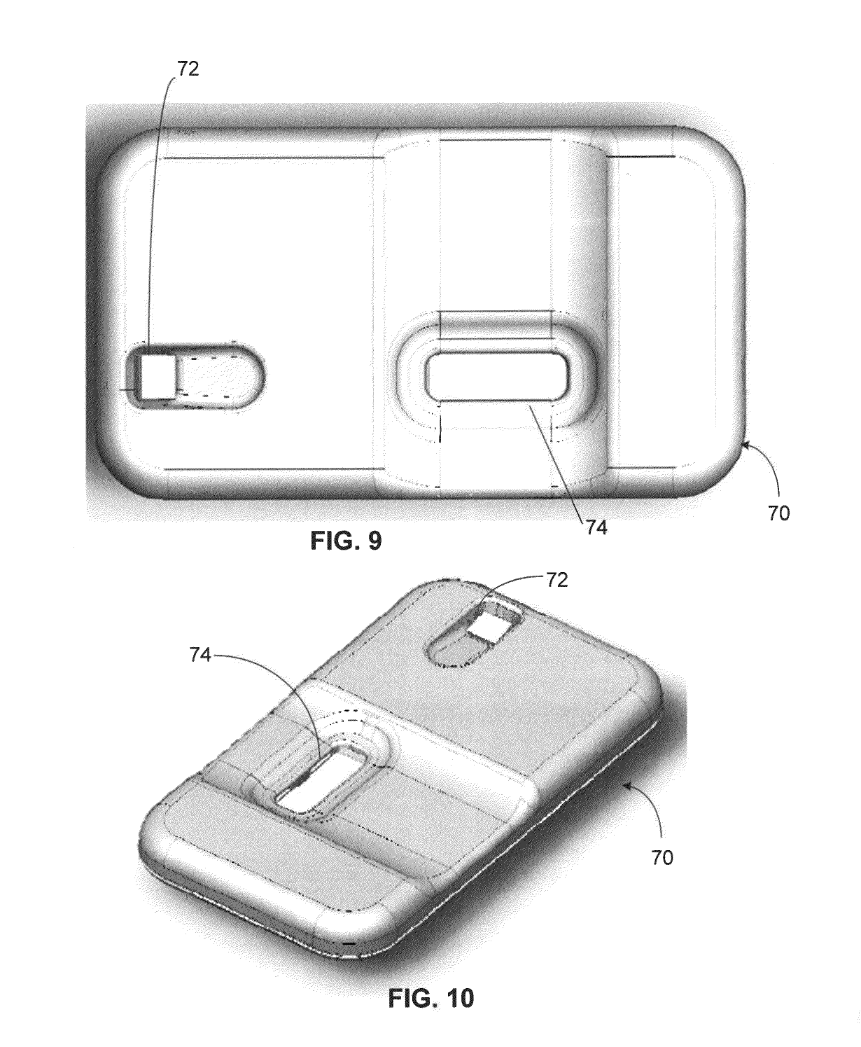

FIGS. 9 and 10 illustrate a cassette 70 which can receive test strip 50. Cassette 70 can comprise a sample receiving well 72 of a predetermined volume and a window 74 whereby the portion of the strip that contains the test line and reference/control line can be exposed. To run the TSH test, a sample of plasma or serum can be placed into sample receiving well 72 (along with any required buffer or other fluid). In some embodiments, the sample well can have a volume of about 45-150 μl.

Another embodiment can include a lateral flow strip configured to detect hTSH by determining an amount of unbound or free thyroxine (FT4) in a plasma or serum sample. T4 is also bound to thyroxine binding globulin (TBG), albumin, and a host of minor protein contributors, which have varying affinities for the hormone. The majority of T4 is bound to TBG (over 99.9%) and is in dynamic equilibrium with free and bound forms. The free form is biologically active, along with free T3, and is thought to therefore be a better indicator of activity over the total T4 (where all the T4 has been removed from the bound proteins and then analyzed). The bound form is subject to removal by a number of chemicals, drugs and physiologic conditions that effect release from the bound form.

The test can operate with a sample volume in the range of 15-50 μl. A pre-mix step can be provided, whereby an equal volume of a dilution buffer can be added to the sample prior to entering the strip. In one embodiment, a dilution buffer of about 30 μl can be added directly to the sample well for premixing with the plasma or serum sample. The sample well can have a barrier (e.g., a pull tab barrier) that prevents the solution from entering the sample pad on the strip until the barrier is removed. Barriers, such as pull tab barriers can assure full sample acquisition, assure sample quantity (no leakage into the system, allowing excess sample—critical when we use sample volume to provide a quantified result), and can control timing (start or total) of the test. A plasma or serum sample of about 30 μl can then be added to the sample well, and the mixture can be gently mixed by pipet action (e.g., stirring) in the well.

Sample well preferably is at least about 100 μl, and preferably about 150 μl to hold both the dilution buffer and the plasma or serum sample for premixing. By eliminating premixing before providing the sample mixture to the cassette (i.e., by allowing premixing to occur in the sample well of the cassette), the test can be more convenient to run and the use of vials or other containers for mixing of the sample and a transfer step can be eliminated.

The dilution buffer can be useful in removing non-specific binding to the capture zone in serum and plasma samples, in removing heterophilic antibody interactions and to aid flow of the sample mixture.

The barrier can then be removed (e.g., by pulling the pull tab), allowing the sample mixture to flow freely into the sample pad. The sample pad can be made of a blood separation matrix (Cytosep, Ahlstrom). This matrix can remove harmful latex aggregation factors in the serum. After the sample mixture flows through the matrix, it arrives at the conjugate pad. The conjugate pad can contain Carboxy Latex impregnanted with a Europium chelate (CMEU, Seradyne, Inc.) that has a biotinylated anti-T4 antibody coated to the surface of the particle. The T4 in the sample mixture reacts with the CMEU-Ab particle to fill available binding sites.

The sample mixture and particles migrate onto the nitrocellulose membrane and toward the primary capture zone (e.g., the reference line), which is an immobilized 1.0 mm band of BSA-T4. The particles will bind or not bind to the primary capture line depending upon how many available binding sites have not been filled.

Those particles leaving the primary capture zone move toward the secondary capture zone (i.e., the test line comprising about 500 ng streptavidin) where the particles containing biotin are captured.

The test can be allowed to clear for about 15 minutes. At that time the cassette/strip can be placed in the optical reader system, where a T/R ratio is calculated from the peak areas of the test and reference lines. That T/R value can then correlated with a stored calibration curve to deliver a test result in pg/ml or ng/dL of FT4.

### Example 2

### FT4 Test with CMEU-Latex-T4 Antibody-Biotin Conjugate and Reader System Reagents

A CMEU Latex-T4 antibody-biotin conjugate was prepared using a modification of the procedure used in example 1, replacing the antibody with anti-T4 monoclonal antibody. The CMEU conjugate is sprayed onto blocked glass at a rate of 1 μL/cm, and dried to form the conjugate strip.

Thyroxine (T4, Neogenesis 707801) calibration test solutions were prepared in a human plasma matrix (American Red Cross, 21KR 78229) by spiking with T4 diluted in stripped serum (Biocell 1131-00) to levels of 0, 75, 175 ng/ml T4. Calibration test solutions T4 level was determined using EIA Kit (Diagnostic Automation Incorporated 3146Z) and found to be 13.3, 31.7, 58.0 pg/mL respectively.

### Construction of Test Strips

A 15 mm by 30 cm strip of nitrocellulose (Millipore SHF1350425) was affixed to adhesive coated vinyl backing (G&L 46166).

One line of a 750 ug/ml solution of the BSA-T4 was applied to (sprayed on) at a rate of 1 μL/cm to the nitrocellulose membrane (primary capture/reference) to produce a 1 mm band. One line of a 1000 ug/ml streptavidin solution was applied to (sprayed on) the nitrocellulose membrane (test/read line) to produce a 1 mm band. The nitrocellulose membrane was dried for 65 hours at 37° C.

A sample pad sheet (Cytosep Ahlstrom 1663) was blocked for 30 minutes in a blocking solution then dried overnight. The dried sample pad was then laminated onto the lower portion of the strip. A cellulose pad (Millipore SA 3J060V04) was laminated to the upper portion of the strip to act as absorbent reservoir.

The CMEU Latex-T4 antibody-biotin conjugate strip is then inserted between the membrane and the sample pad with a 3 mm overlap onto the membrane.

A plastic liner strip 4.3 mm×15 mm (G& L 46166) was cut and placed over the sample well opening of the cassette to act as a sample barrier pull tab.

The completed card was cut into 4.3 mm strips. Strips were inserted into plastic cassettes with a hole/well located in the sample application area and a window in the test reading area.

### Test Procedure

Samples used were thyroxine (T4, Neogenesis 707801) spiked into a human plasma matrix (American Red Cross, 21KR 78229) by spiking with T4 diluted in stripped serum (Biocell 1131-00) to levels of 50 and 125 ng/mL T4. Sample solutions FT4 level were determined using EIA Kit (Diagnostic Automation Incorporated 3146Z) and found to be 21.4 and 44.0 pg/mL respectively.

A 60 ul aliquot of the sample and a 60 ul aliquot of FT4 test solution was added to the sample well of each test strip device and mixed with a pipet. The sample well barrier tab was pulled, allowing the solution to be absorbed by the sample pad. The test strips were allowed to develop for 15 minutes.

### Results

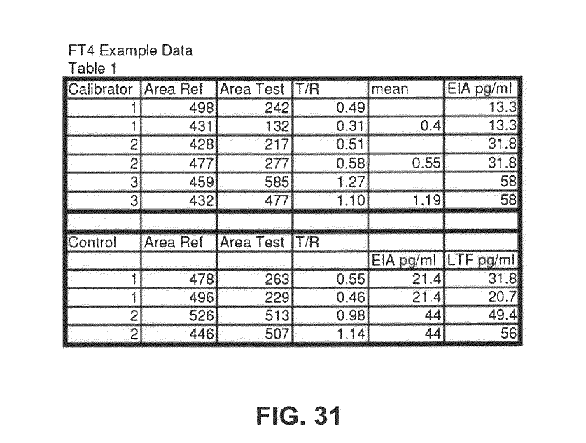

The test strips were then quantitated using the reader system to obtain values for the area of the reference and test lines. The T/R ratio was calculated from the areas, and the calibration curve was established. The results are depicted in Tables 1 in FIG. 31.

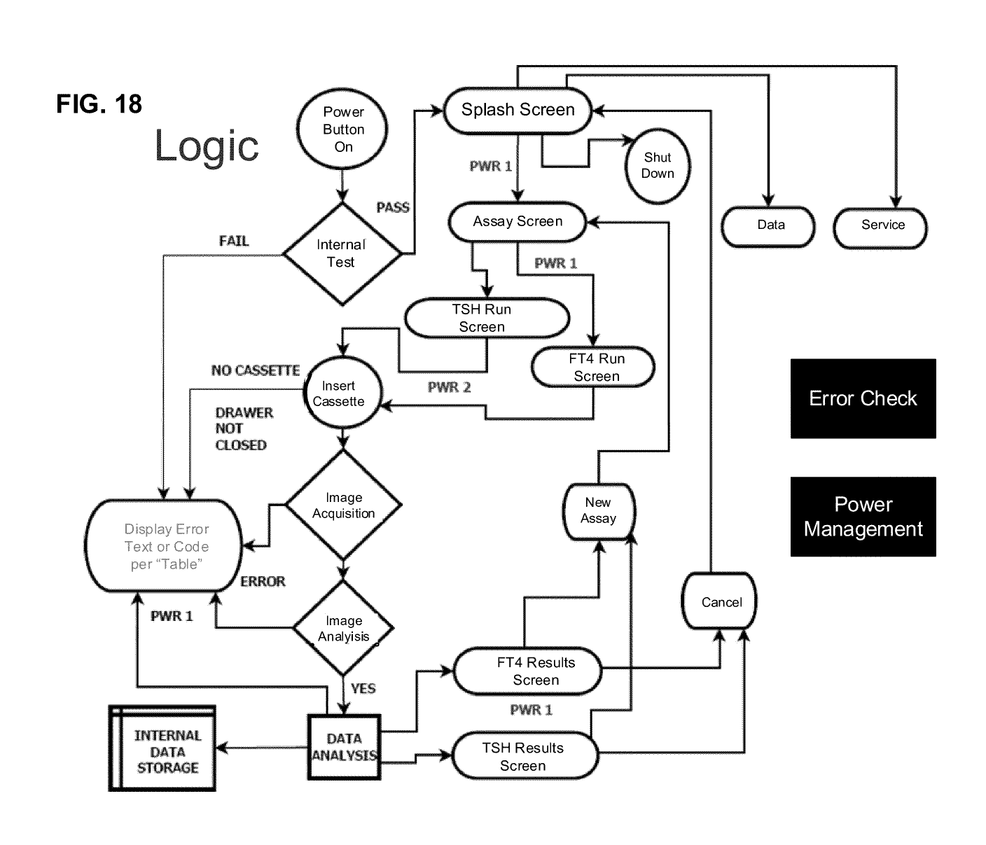

Referring to FIG. 18, a flow chart illustrates various steps that can be performed by reader system 10 in connection with the TSH test disclosed above.

The above examples illustrate two embodiments of lateral flow assays that can used in combination with the optical reader systems disclosed herein. However, it should be understood that the use of the optical reader systems (or the use of portions of the optical reader systems) disclosed herein are not limited to such embodiments. The optical reader systems disclosed herein can be used in combination with various lateral flow assays, including sandwich- and competitive-type assays such as those disclosed in U.S. Pat. No. 6,699,722. U.S. Pat. No. 6,699,722 is incorporated by reference herein in its entirety.

Depending on the structure and materials of the lateral flow assay, the optical reader can be adapted to excite and detect various labels that have been captured, or are otherwise present, in lines or areas in a lateral flow assay to provide a quantitative measurement result of the amount of analyte in a sample. In some cases, the quantitative measurement result can take into consideration a ratio of a measured response in two zones (e.g., a test band to a reference band).

In other cases, however, only a single zone can be detected and/or no ratio need be determined in connection with the provision of the quantitative measurement result.

### Excitation Source

Fluorescence detection is commonly used in highly sensitive assay detection or imaging systems, for example biomedical diagnostic or analytical or research devices. Conventional fluorescence detection uses wavelength filtering to isolate the shorter-wavelength excitation photons from the detected longer-wavelength emission photons, since the detected photon flux is typically many orders of magnitude lower than the excitation photons. Sensitive assay systems can require expensive optical filters, and also require careful selection of assay substrate materials, so that autofluorescence from these materials does not interfere with the desired fluorescence signal. Commonly, very low cost, disposable materials are desirable for such assay devices, but it is difficult to find such materials that do not interfere with the fluorescence detection.

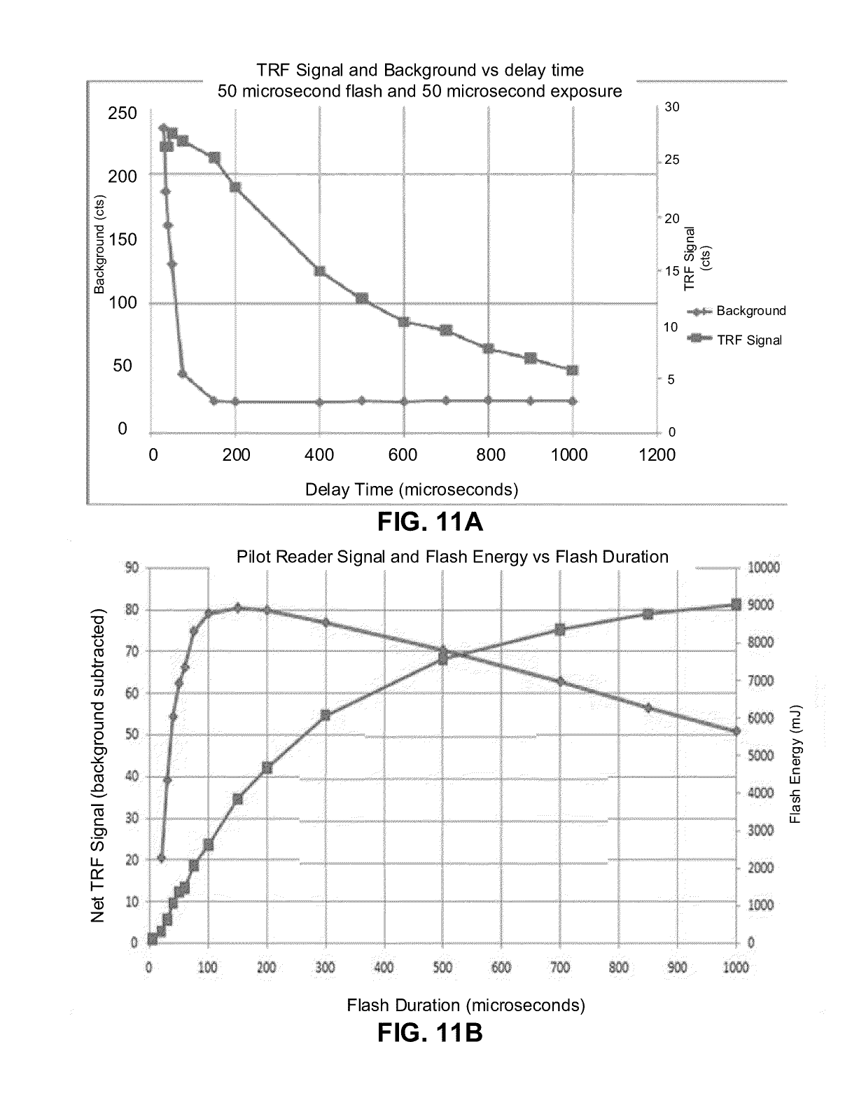

Time resolved fluorescence (TRF) is a powerful detection technique that utilizes fluorescent tags with long emission lifetime, which solves some of the challenges above. In TRF, brief pulses of light can be used to excite the fluorescent labels or tags, which continue to emit fluorescent signals after the pulse is terminated, typically for times from a few microseconds to hundreds of microseconds. After the pulse is terminated, and after an additional delay period, the detection system is triggered to measure the long-lived TRF signal. This is advantageous for the applications described above, because wavelength filtering is sometimes not required at all (or can use lower-performance lower-cost filters) since the detection system measures the signal after the excitation energy has been removed. It is also advantageous because the autofluorescence from materials used in the assay device typically has fluorescent lifetime in the range of nanoseconds, and this source of background and noise is essentially entirely eliminated before the detection system begins to measure the TRF signal. FIG. 11A is a chart showing a measured TRF signal relative to a measurement of background noise over time.

High performance, low cost flashlamps have been developed for consumer products such as digital cameras and smart-phones. These lamps have very broad spectrum emission, even in the ultraviolet, and high output energy (multiple Joules per flash). In certain embodiments of the reader systems disclosed herein, the excitation member can be a flashlamp that can emit enough excitation energy in a single flash to perform fluorescence or TRF measurements with adequate sensitivity (as opposed to averaging many flashes together to increase the sensitivity). By using flashlamp technology, a reader system can be provided that is simple and relatively low-cost.

By utilizing a flashlamp as excitation member 24, only a brief pulse of light is needed to excite the signal from a TRF tag or label, so it is possible to design a system that uses time discrimination instead of wavelength filtering, as described above. Such a system can work without any optical filters at all, thereby reducing both cost and complexity of the system. However, high energy flashlamps heat up significantly during the brief flash duration (which can require hundreds of volts and hundreds of amperes of current, even for short durations). After the flash, the hot lamp continues to emit blackbody radiation for a period of time as it cools to ambient temperature. During that time, the blackbody radiation may contain enough energy at the detection wavelength to interfere with the TRF signal measurement. Therefore, as shown in FIGS. 2 and 4, it can be advantageous to include an optical filter 30, such as an excitation short-pass or band-pass filter) between the lamp and the detection zone.

Optical filter 30 can pass the desired wavelengths that excite the fluorescent labels and block the longer wavelengths from blackbody radiation emitted by the hot lamp, even after the flashlamp current is terminated. Thus, optical filter 30 can have the effect of greatly reducing unwanted background radiation that would interfere with the measurement. Such a filter is typically much lower cost than the high-performance filters that are required for fluorescence systems with low Stoke's shifts, such as the interference filters that are used in conventional fluorescence detection systems to discriminate between the excitation and emission wavelengths.

Flashlamps are energized by circuits using high voltage and high currents, to generate high energy flashes with short duration, measured in the range of a few microseconds to hundreds of microseconds. TRF tags and labels, such as lanthanide chelates of europium, emit fluorescence over a time range of tens of microseconds to milliseconds. To optimize the measurement sensitivity of a TRF system, it is important to control the flashlamp duration and begin the signal detection at the optimum time to maximize TRF signal.

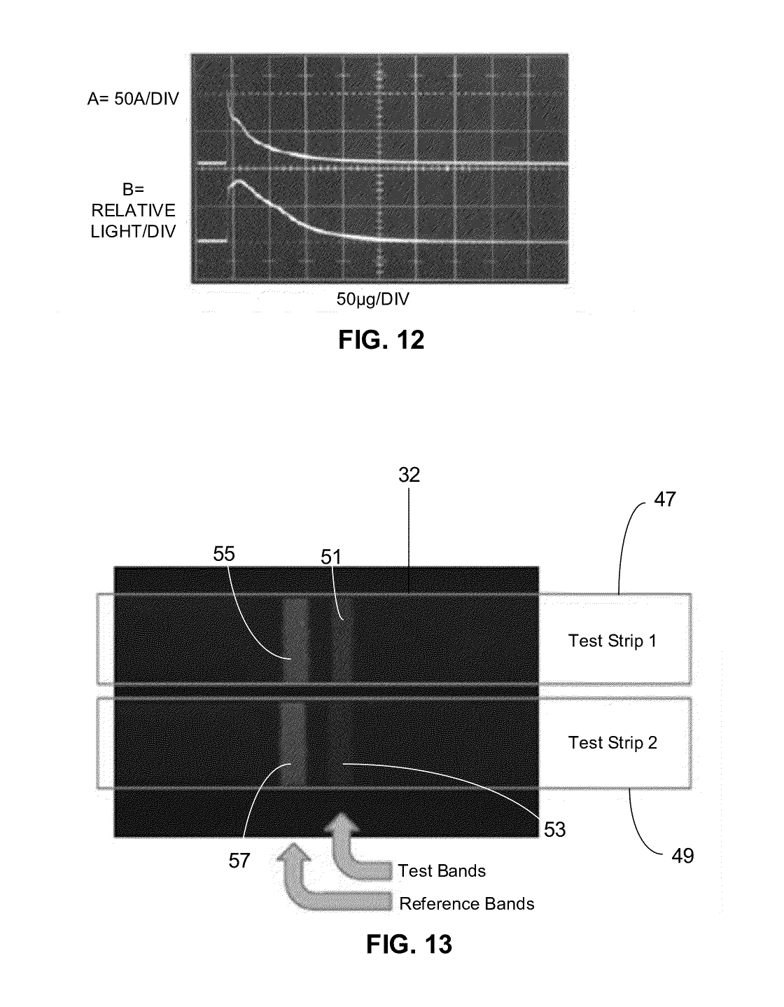

Flashlamp circuits can charge up a capacitor to a high voltage (typically a few hundred volts), and then direct that charge to flow into the flashlamp. The flashlamp's light emission peaks shortly after the current begins to flow, and then gradually decays (similar to an RC exponential decay curve) over a period as long as hundreds of microseconds or even milliseconds, depending on the capacitance and flashlamp impedance, as shown in FIG. 12. FIG. 12 illustrates a photograph capture of the entire current (Trace A) and light events (Trace B), with the waveform leading edges of FIG. 12 being enhanced for clarity. The light output generally follows the current profile, although peaking is less defined. In order to maximize the TRF signal from fluorescent labels such as europium chelates, it may be important to terminate the flashlamp current before the charge capacitor has completely dissipated, because the TRF measurement cannot begin while the flashlamp is still emitting light.

It is advantageous to include a high-current switching transistor in series with the flashlamp, so that the flashlamp can be turned off at the optimum time, so that more of the TRF signal can be captured by the detector. For example, the flashlamp flash duration shown in FIG. 11A is 200 microseconds or more, though most of the flashlamp energy is delivered in the first 50 microseconds. By terminating the flash after about 100 microseconds, much more of the TRF signal (shown by the upper curve in FIG. 11A) can be captured by the detector.

FIG. 11B illustrates a comparison of TRF signal and flash energy relative to flash duration. As shown in FIG. 11B, a maximum TRF signal can occur with a flash duration of about 100 to 200 microseconds. To limit flash energy, however, it can be preferable to choose a flash of less than about 150 microseconds, and, more preferably, of about 100 microseconds. At 100 microseconds, the flash energy is estimated to be about 2500 mJ, which can desirable result in a longer flashlamp lifetime (e.g., about 10,000-20,000 flashes).

### Multiple Analytical Tests

As discussed in more detail below in other embodiments, field of view 37 of the imaging system can be sufficiently large to detect multiple portions of different analytical tests or test members. As shown in FIG. 13, these multiple portions can include, for example, portions of multiple lateral flow strips 47, 49 (or other analytical test members). FIG. 13 illustrates a detection zone 32 of an imaging system that includes portions of two lateral flow strips 47, 49. The portions of two lateral flow strips 47, 49 that are within detection zone 32 include test bands 51, 53 and reference bands 55, 57, respectively. Accordingly, detection zone 32 can be sufficiently large to read the relevant test and reference bands of the two lateral flow strips 47, 49 shown in FIG. 13.

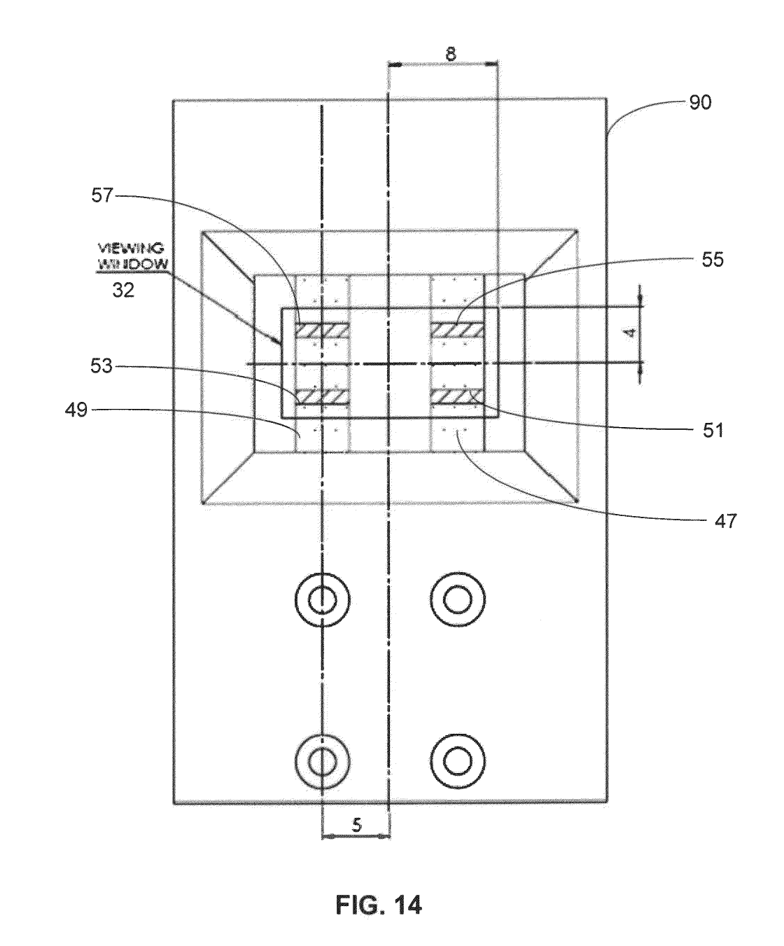

Preferably, lateral flow strips 47, 49 can be included in a single cassette to facilitate loading of the two lateral flow strips into reader system 10. For example, FIG. 14 illustrates lateral flow strips 47, 49 positioned within a cassette 90. As shown in FIG. 14, detection zone 32 can be sufficiently large to encompass the test and reference bands of lateral flow strips 47, 49.

In other embodiments, more than two lateral flow strips can be loaded into reader system 10 for simultaneous and/or sequential analysis using the imaging systems described herein. FIG. 14 illustrates four lateral flow strips 61, 63, 65, and 67. Once positioned into reader system 10, each of these lateral flow strips has a portion that falls within detection zone 32 (e.g., a reading window of the imaging system). Again, as described above, the portions of the lateral flow strips that fall within the detection zone 32 include test and reference lines as shown in FIG. 14. As described in FIG. 14 with regard to lateral flow strip 61 can include a sample pad 71, a conjugate pad 73, a membrane 75, and an absorbent pad 77. For clarity, the relevant portions of lateral flow strips 63, 65, and 67 are unlabeled in FIG. 14. However, it should be understood that those lateral flow strips can be generally similar to lateral flow strip 61.

### Additional Information Obtainable Using Image Sensors

As noted above, the imaging systems described herein can also be used to image other information present in the field of view of the imaging system. For example, bar-code labels are frequently used in assay systems to provide calibration or lot-specific information that is required to increase the sensitivity or precision of the system. In conventional systems, this information must be read by a specific bar-code reader. In the systems described herein, however, the imaging system can read the bar-code information along with the fluorescent or other signals associated with the assay test itself.

In particular, many assay devices and systems use calibration techniques to correct for lot-to-lot variations in the disposable cassettes. For example, each batch of manufactured cassettes may have different performance, which requires the reader to correct the measurements from those cassettes using well-known techniques such as standard curves to convert measured signals to reported analyte concentrations. Such systems require the user to enter such lot-specific calibration information, such as the parameters defining a standard curve, into the system for each measurement. Often, bar-code readers or radio-frequency identification (RFID) systems are used to automatically transfer such information into the system, saving labor for the user.

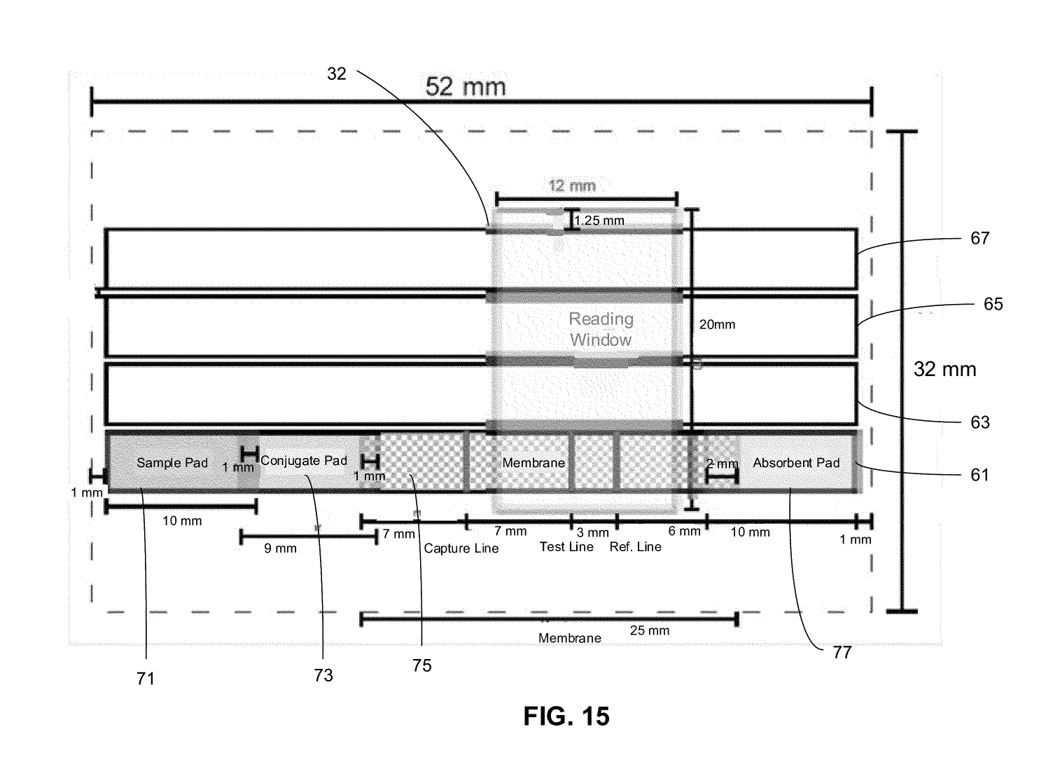

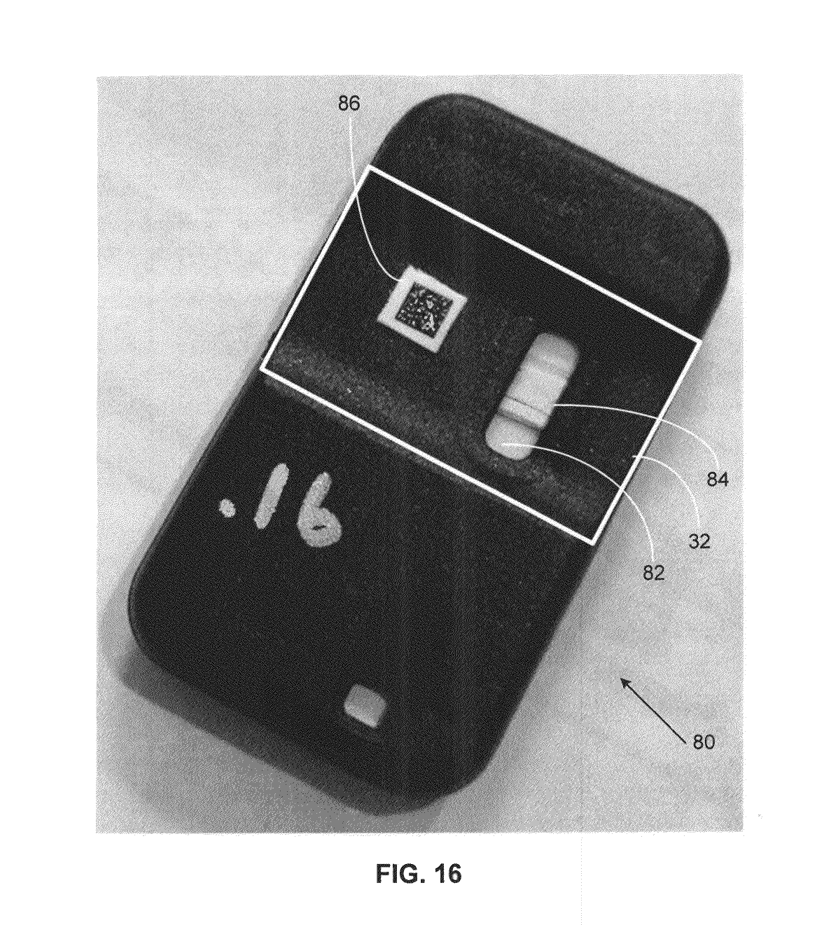

A further advantage of using an imaging system as described herein is that the imaging system can also be used to image a barcode label, integrated with the sample cassette, which can reduce time and cost associated with a separate code reading component, such as a barcode reader or RFID system. FIG. 16 illustrates a cassette 80 that comprises at least one lateral flow strip 82. As shown in FIG. 15, detection zone 32 of the imaging system is sufficiently larger to capture data from a window 84 in cassette 80 (to read information from lateral flow strip 82) and to capture date from a bar code member 86.

Data on the bar code member can include for example a test identifier (e.g., TSH, FT4, TCP, Opiates, etc.), a production lot code, a date of manufacture or update, one or more codes that allow or instruct the reader to adjust the test parameters so that consistent readings are obtained from reader-to-reader and lot-to-lot, and any other information that is necessary or useful for consistent operation of the system. For example, data that define calibration settings that may vary from lot-to-lot can include slope coefficients or spline fit values, camera control variables such as for exposure time, lot codes, test types, antipiracy schemes.

Two examples of such data dense barcodes are Code128, and DataMatrix. The bar codes can be printed directly on the test (cassette) housing or onto a label, which can then be affixed to the cassette. The bar codes can be located in a position that can be read by the reader's optical system, such as adjacent a window of a cassette as shown in FIG. 16. The barcode can be illuminated by the flashlamp discussed above to render the barcode easily visible to the imaging system (e.g., a cmos sensor) and decodable by software. Alternatively, a separate illumination member, such as a white LED can be used to illuminate the barcode.

### Cassette

Lateral flow strips are typically constructed with several layers of materials, intended to channel sampled fluids, such as blood, serum, plasma, urine, oral fluid, vaginal fluids, or collected extracts of the same, diluted or mixed with other fluids, such as buffer, conjugate, or diluents. These layers are usually made from fibrous or non-woven materials, used to separate red blood cells from plasma, particulate from urine or oral samples, or to place dried conjugate in the fluid pathway created by this construct. A significant problem with these methods is that the ends of the layers, or pads, can become loosened, and create a blockage to liquid flow.

Typically, lateral flow strips are encased in a plastic package or cassette that is formed of PVC or another plastic material. The cassette generally includes thin internal walls that press on key locations along the lateral flow strip. These thin walls are usually referred to as pinch points. Pinch points are also critical to control the rate of flow in the lateral flow strip, and, at the sample and, if needed, buffer port, to surround the port to control the flow of fluid(s) into the membranes the make up the device.

Such pinch points, however, can create other problems. In particular, if the pinch points are too tight, they can cause a blockage of flow, and if too loose, they can cause a flow blockage at the end of the membrane they are intended to assist, or allow fluid to enter the cassette in an uncontrolled flood. Since pinch points are part of the molded cassette, they are subject to manufacturing and material variations that are beyond reasonable means of control.

The novel cassette devices described herein reduce and/or eliminate the deficiencies described above with conventional pinch points, while allowing optimal control of the pinch points regardless of the manufacturing or material variations.

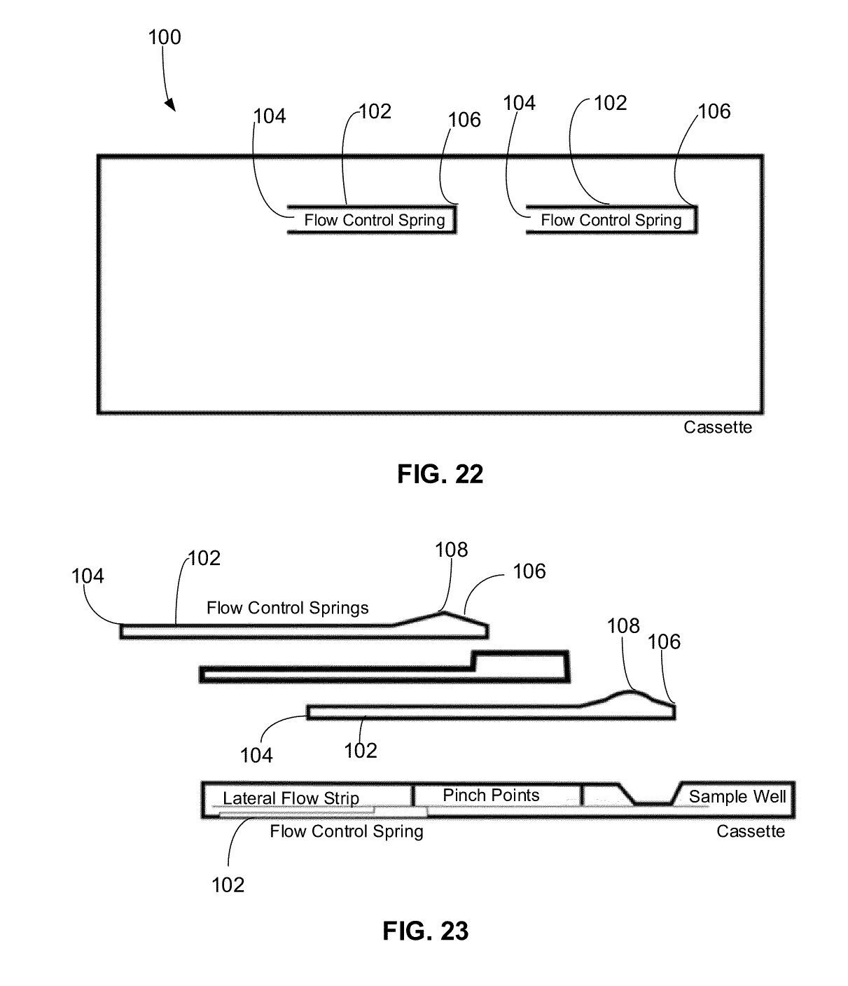

As shown schematically in FIG. 22, a cassette 100 can comprise one or more biased members 102 configured to provide a force against a lateral flow strip contained in cassette 100. As shown in FIG. 23, biased members 102 can comprise flow control springs that are positioned on the back of cassette 100. Such biased members 102 can be cantilevered members that have a fixed end 104 (e.g., an end coupled to the cassette 100) and a free end 106.

Referring again to FIG. 23, free end 106 of biased member 102 can comprise a protuberance 108. Protuberance 108 can be configured to gently press upon the back of the lateral flow strip within cassette 100, and using the natural flexibility of the cassette material, provides the gentle pressure required for proper liquid flow within the lateral flow strip. Protuberance 108 can be square, round, triangular, or other appropriate shape.

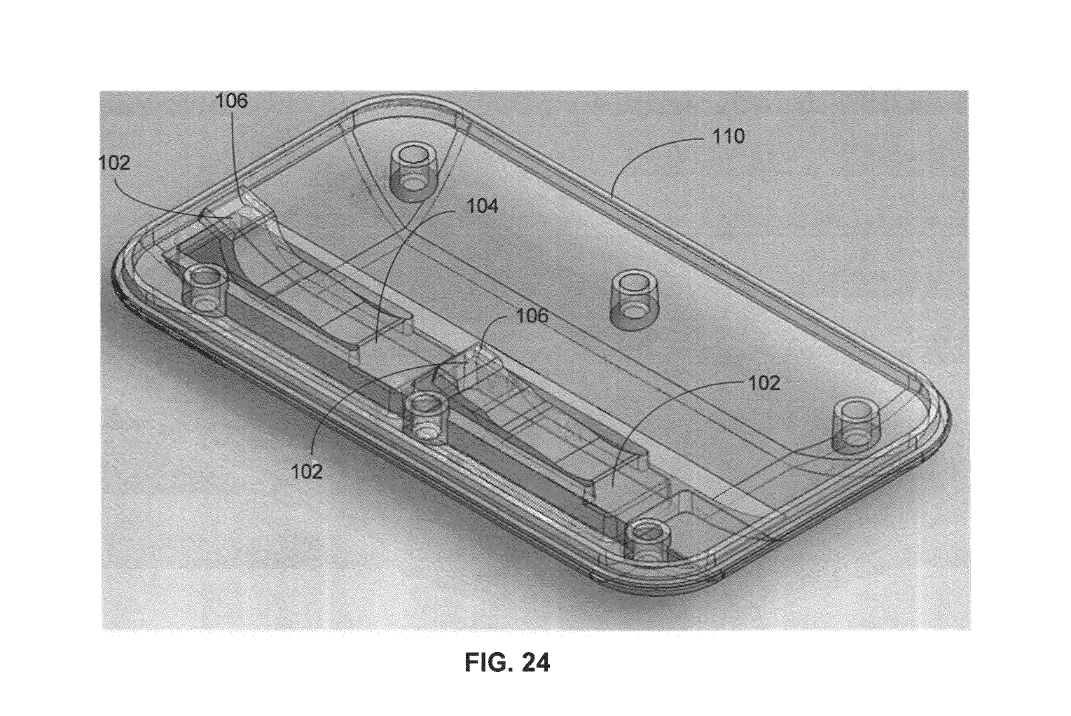

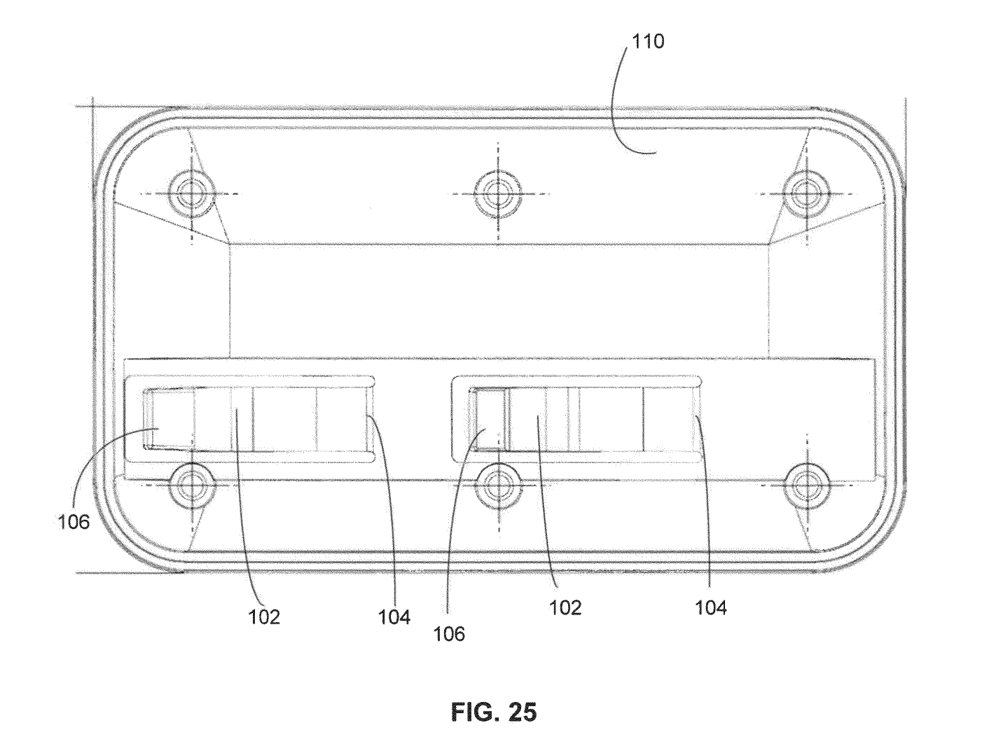

FIGS. 24 and 25 illustrate a bottom member 110 of cassette 100, which includes two biased members 102 that are configured to exert an upward force on a lateral flow strip contained in the cassette. The biased members can be configured to exert between about 30 and 400 grams of force to the back of the lateral flow strip and, more preferably, between about 30 and 300 grams of force.

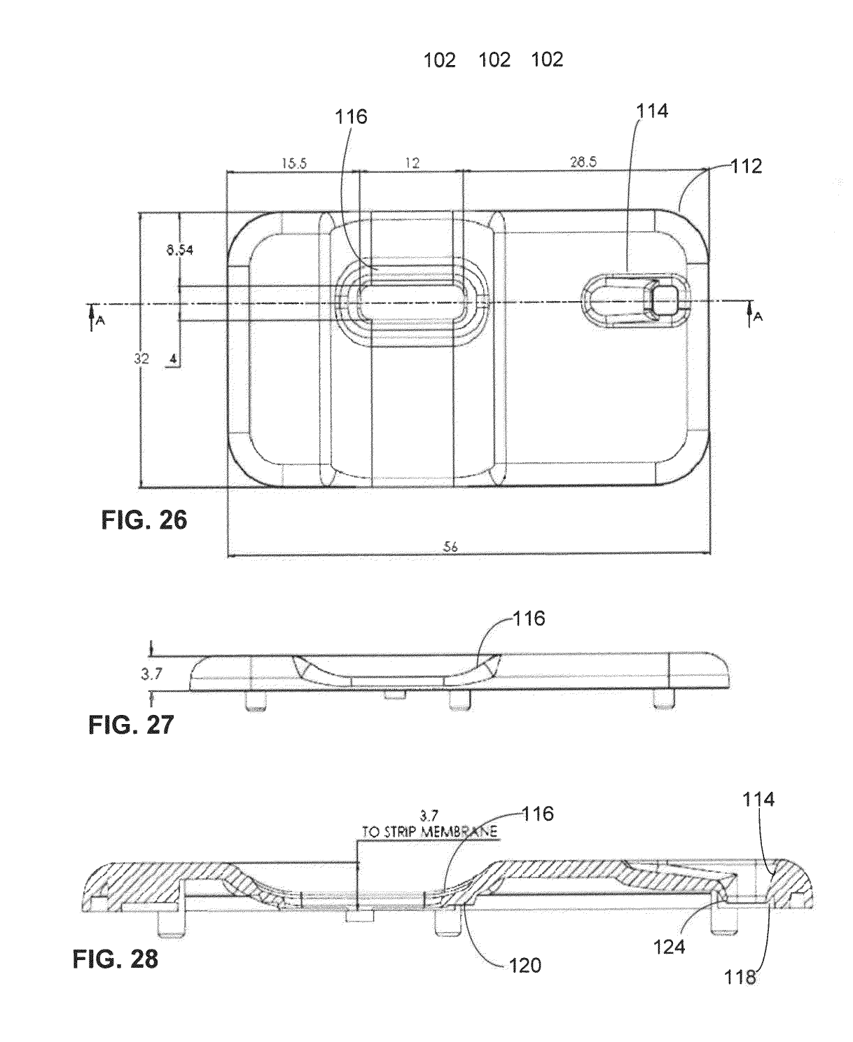

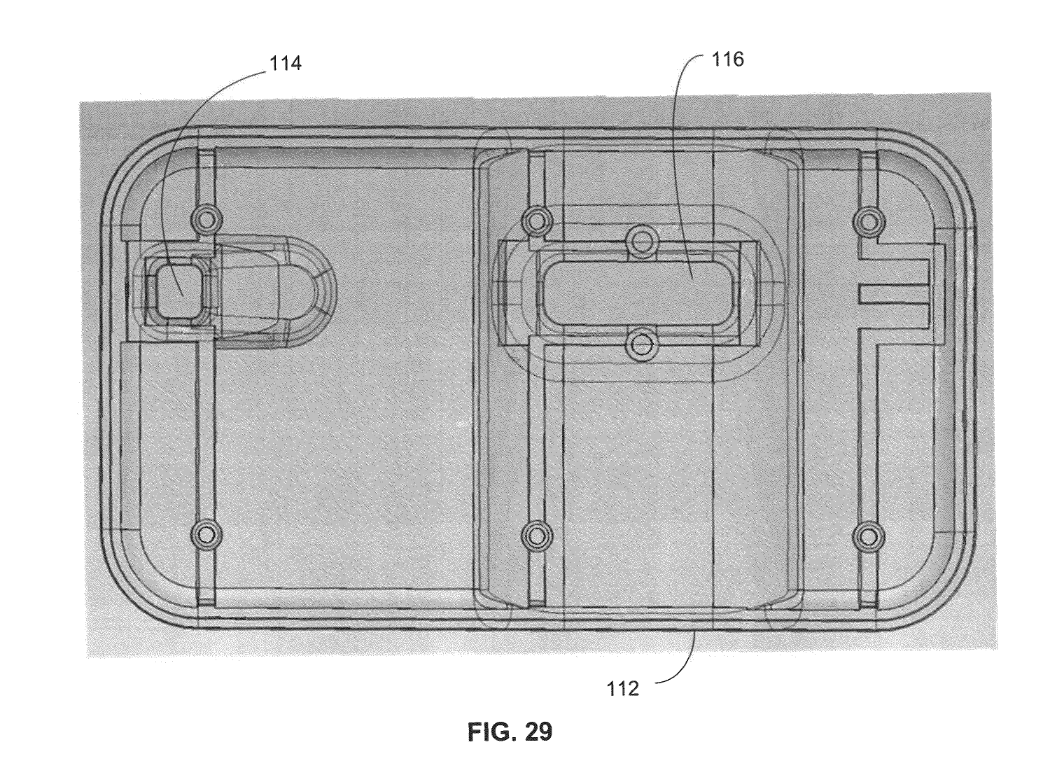

FIGS. 26-29 illustrate a top member 112 of cassette 100, which includes a sample well 114 and a viewing window 116. Viewing window 116 is preferably recessed as shown most clearly in FIG. 27. By recessing window 116 in this manner, excitation light can be more easily directed at window 116 (which is positioned in the detection zone) and the amount of shadows of other interfering structural elements can be reduced. Referring again to FIG. 23, a first pinch point 118 can be provided adjacent sample well 114 by contact with a first biased member 102 and a second pinch point 120 can be provided adjacent window 116 by contact with a second biased member 102. The biased member 102 in contact with the pinch point 118 adjacent sample well 114 can be configured to apply a greater amount of pressure to the lateral flow strip than the other biased member, since it is desirable to prevent flow of the sample mixture in the direction upstream of sample well 114.

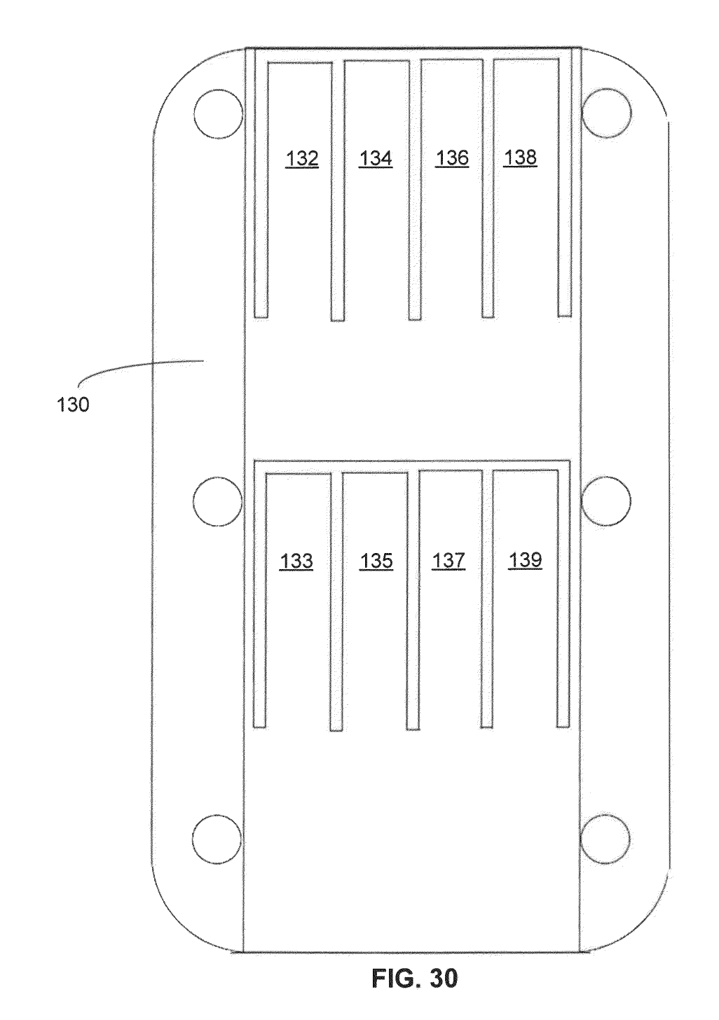

FIG. 30 illustrates an embodiment with more than two biased members. As discussed above, multiple lateral flow strips can be provided in a single cassette. FIG. 30 illustrates a back of a cassette 130 that comprises four pairs of biased members 132 and 133, 134 and 135, 136 and 137, and 138 and 139.

The cassettes disclosed herein should be sized to fit the lateral flow strip being housed therein. In some embodiments, cassettes of the present disclosure can be between about 40 and 80 mm long and, more preferably, between about 50 and 60 mm; between about 20 and 45 mm wide and, more preferably between about 25 mm and 40 mm wide; and between about 5 and 20 mm in height and, more preferably, between about 5 and 12 mm in height. In one embodiment, a cassette is about 56 mm long, 32 mm wide, and has a height of about 8 mm.

A window (e.g., window 116 of FIG. 26) is preferably large enough to allow the results of a lateral flow assay to be read by an optical reader as described herein. Preferably, the window is between about 5 and 20 mm long and between about 2 and 10 mm wide; however, other sizes can be selected depending on the area that is intended to be viewed. In one embodiment, the cassette window can be about 12 mm long and 4 mm wide. As described above (e.g., FIG. 16), the viewing area of the optical reader can comprise more than just the window area of the cassette.

The window can also have a depth, such as that shown in FIGS. 7, 27, and 28. As shown in FIG. 7, the window depth can facilitate the delivery and/or receipt of light to and/or from the strip. For example, as shown in FIG. 7, an excitation member 24 (e.g., a flashlamp) can be provided in an off-set orientation (e.g., a non-normal orientation) and light from the excitation member 24 can reach a greater portion of the strip since the cassette has an angled (or recessed) area. In one embodiment, the depth of the window can be between about 1 mm and 3 mm (e.g., about 2 mm).

The biased members described herein are preferably positioned on the bottom portion of the cassette as shown in the figures; however, it is possible to place them on a top portion. Also, one or more microfluidic channels can be provided in the cassette to restrict or direct flow of the sample mixture in a desired direction. Thus, for example, one or more microfluidic channels can be provided at or adjacent pinch points 118, 120 to reduce pressure on the lateral flow strip and cause fluid flow to move through the test strip and the microfluidic channels in the desired direction. In one embodiment, a wall member 124 comprises microfluidic channels that reduce pressure and encourage flow of the sample mixture through the channels of wall member 124.

In view of the many possible embodiments to which the principles of the disclosed embodiments may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of’ excludes any element, step, or ingredient not specified in the claims. The transition term “consisting essentially of’ limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A cassette comprising:
a housing comprising a top member and a bottom member; and
a lateral flow strip receiving area located between the top and bottom members;
wherein the housing comprises one or more biased members that have a fixed end and a free end, the free end being configured to contact at least a portion of a lateral flow strip when the lateral flow strip is positioned in the lateral flow strip receiving area.

2. The cassette of claim 1, wherein the at least one biased member is formed in the bottom member.

3. The cassette of claim 2, wherein the at least one biased members comprise two biased members.

4. The cassette of claim 3, wherein the two biased members extend along the length of the cassette and are generally in-line with one another.

5. The cassette of claim 4, wherein the biased members exert a force of between about 30 and 400 grams on the back of the lateral flow strip.

6. The cassette of claim 1, wherein the housing comprises a plurality of lateral flow strip receiving areas, and each lateral flow strip receiving area is configured to receive one lateral flow strip.

7. The cassette of claim 1, comprising a plurality of biased members and wherein the free end of each biased member is configured to contact at least a portion of one lateral flow strip when the lateral flow strips are positioned in the plurality of lateral flow strip receiving areas.

8. The cassette of claim 1, wherein the top member comprises at least one window, the window exposing a portion of the lateral flow strip when the lateral flow is positioned in the lateral flow strip receiving area.

9. The cassette of claim 8, wherein the window is at least partially recessed.

10. The cassette of claim 8, further comprising coded information positioned on a surface of the top member adjacent the window.

11. The cassette of claim 8, wherein the coded information comprises lot-specific calibration information.

12. The cassette of claim 1, further comprising a lateral flow strip positioned in the lateral flow strip receiving area.

13. The cassette of claim 12, further comprising a sample well for receiving a test sample.

14. The cassette of claim 13, wherein the sample well has a volume of between about 45 and 150 microliters.

15. The cassette of claim 14, further comprising a pull barrier between the sample well and the lateral flow test strip.

16. The cassette of claim 12, wherein the lateral flow strip comprises a TSH test strip.

17. The cassette of claim 12, wherein the lateral flow strip comprises a FT4 test strip.
